(19) United States
(12) Patent Application Publication
(10) Pub. No.: US 2024/0409724 A1
(43) Pub. Date: Dec. 12, 2024

(54) CHITOSAN-BASED SWELLING GEL

(71) Applicants: MEXBRAIN, Villeurbanne (FR); UNIVERSITE CLAUDE BERNARD LYON 1, Villeurbanne Cedex (FR); CENTRE NATIONAL DE LA RECHERCHE SCIENTIFIQUE, Paris (FR); INSTITUT NATIONAL DES SCIENCES APPLIQUEES DE LYON, Villeurbanne (FR); UNIVERSITE JEAN MONNET SAINT ETIENNE, Saint Etienne (FR)

(72) Inventors: Olivier TILLEMENT, Fontaines Saint-Martin (FR); François LUX, Villeurbanne (FR); Thomas GREA, Lyon (FR); Coralie GRANGE, Villeurbanne (FR); Axel AIGLE, Bron (FR); Anouk ROUDIER, Salaise sur Sanne (FR); Laurent DAVID, Lyon (FR); Alexandra MONTEMBAULT, Rive-de-Gier (FR); Augustin TILLEMENT, Fontaines Saint-Martin (FR); Renaud PASSIEUX, Villeurbanne (FR); Juan Felipe SALAZAR ARIZA, Bogota (CO)

(21) Appl. No.: 18/698,471

(22) PCT Filed: Oct. 3, 2022
(86) PCT No.: PCT/FR2022/051864

§ 371 Date: Apr. 4, 2024

(30) Foreign Application Priority Data
Oct. 4, 2021 (FR) FR2110474

(51) Int. Cl. C08L 5/08; A61K 47/36; A61L 15/28; A61L 15/60; A61L 24/00; A61L 24/04; A61L 31/04; A61L 31/14

(52) U.S. Cl. CPC C08L 5/08; A61K 47/36; A61L 15/28; A61L 15/60; A61L 24/0031; A61L 24/043; A61L 31/042; A61L 31/145; C08L 2203/02; C08L 2203/12; C08L 2205/025

(57) ABSTRACT

The present invention relates to the field of compositions comprising a polymer bearing a chelating group capable of chelating one or more metals, and to the various uses thereof. In particular, the present invention relates to compositions comprising a copolysaccharide carrying a chelating group, which compositions may be in the form of a gel, as well as to the threads obtained from this gel.

### TECHNICAL FIELD

The present invention relates to the field of compositions comprising a polymer bearing a chelating group which can chelate one or more metals, and to the various uses thereof. In particular, the present invention relates to compositions which may be in gel form, comprising a polysaccharide bearing a chelating group, and also to the threads obtained from these gels.

### PRIOR ART

Polysaccharides are polymers derived from plant, fungal, animal or bacterial biomass. These polymers have highly varied physico-chemical properties and can be used in a broad range of biomedical applications because they are often resorbable, biocompatible or even bioactive. The chemical modification of polysaccharides makes it possible to adapt the physico-chemical properties thereof, in particular the solubility thereof in aqueous medium at near-neutral pH. The functionalization of polysaccharides by highly-specific chelating agents also enables particular applications in the biomedical field. For example, after grafting these units to the polysaccharide structure, the polymer could be used in the composition of a detoxifying biomedical device for removing pathogenic metals from living organisms, such as in the context of maintaining homeostasis.

Maintaining homeostasis of the internal environment of the body, i.e. all of the biological liquids or fluids of the body, is necessary for the proper functioning of said body. Systemic or local dysregulations in metal homeostasis have been demonstrated for a number of diseases. Chelation therapies, which aim to reduce the concentration of metal ions, have already been used for many years in cases of acute intoxication. Thus, a certain number of chelators are already accepted for humans, each of which is associated with a particular group of metals (G. Crisponi et al., Coordination Chemistry Reviews, 2015).

Increasing numbers of scientific studies highlight the important role that metals might play in many neurological disorders, in particular iron but also copper, zinc, manganese, and even aluminum and lead (E. J. McAllurn et al., J. Mol. Neurosci., 2016).

Thus, there are already medical devices which can be used for capturing metals. Nevertheless, it is still of benefit to improve the results obtained to date, in particular to reduce the side effects of these treatments.

Moreover, it is also desirable to have a variety of shapes for medical devices, for example a single-stranded or multi-stranded thread which can be woven or knitted in the form of a textile and which makes it possible to obtain a medical device of the dressing or parietal implant type. A single thread may also be advantageous in the event that the implantation has to be combined with a minimally invasive strategy. Providing a thread which makes it possible to capture metal cations in order to locally restore metal homeostasis when there is an accumulation of metals in the body would therefore be of particular interest.

Thus, one aim of the invention is to propose a composition which can chelate one or more metals. Another aim of the invention is to provide a thread which can chelate one or more metals. Another aim of the invention is to provide a thread having good mechanical properties and which can thus be woven or knitted in the form of a textile.

### SUMMARY

Firstly, the invention relates to a composition comprising:
a. at least one chitosan A, and
b. at least one statistical copolysaccharide B having a weight-average molecular weight of between 100 kDa and 1000 kDa, of formula 1:

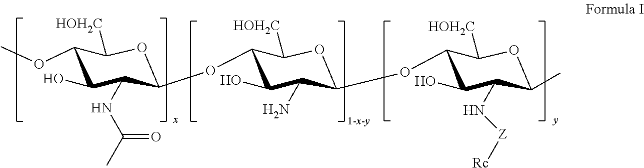

in which:
each Rc independently represents a group comprising a chelating agent,
each Z independently represents a linker which may be a single bond or a hydrocarbon-based chain comprising between 1 and 12 carbon atoms, it being possible for said chain to be linear or branched and to comprise one or more unsaturations and to comprise one or more heteroatoms, preferably selected from nitrogen, oxygen, sulfur and atoms of the halogen family,
x is between 0.01 and 0.5,
y is between 0.05 and 0.5.
the y/x ratio being greater than 0.2, preferably greater than 1,
the sum x+y being greater than 0.1, and
less than 10% of groups comprising a chelating agent of Rc type which are chelated by a cation of a transition element selected from block d or f elements, and
c. optionally water.

The presence of a chelating group of Rc type in the copolysaccharide B enables this composition to efficiently capture one or more metals, and in particular to capture metal cations. Moreover, the use of chitosan A and of polysaccharide B, which is produced from chitosan, makes it possible to obtain a composition with beneficial properties. Indeed, this composition can be biodegradable, biocompatible and bioresorbable. The use of such a composition which makes it possible to chelate metals therefore has numerous possible medical applications.

In addition, it is possible to form a thread from this composition, in particular by extrusion of the composition. This thread has good mechanical properties and can be shaped. It is possible to braid the threads, for example in order to form a (knitted or woven) textile material that is capable of capturing metals.

Moreover, surprisingly, the inventors have demonstrated that this composition was able to swell on contact with a biological fluid to a significant extent. This swelling makes it possible to promote the diffusion and capture of the metal species to be captured, and therefore to improve the efficacy of the material.

The invention also relates to a thread comprising a gel or a lyophilizate of the composition according to the invention.

The invention also relates to a textile material comprising threads according to the invention.

The invention also relates to a medical device comprising the composition according to the invention, the medical device preferably being a dressing, an implant, or a dermal filler product.

The invention also relates to the composition according to the invention, for use thereof for capturing at least one metal, preferably for use thereof for capturing at least one metal in the treatment or prevention of endometriosis, fungal infections, bacterial infections, chronic wounds, neurodegenerative disorders, nervous system lesions, hemochromatosis, Wilson's disease or lead poisoning.

The invention also relates to a method for preparing a composition according to the invention, comprising the following steps:
1) dispersing the chitosan A and the statistical polysaccharide B having a weight-average molecular weight of between 100 kDa and 1000 kDa of formula I in water,
2) adding an acid, preferably acetic acid,
3) mixing then centrifuging the solution obtained in order to recover a solution comprising the composition of the invention,
4) optionally treating the solution obtained in step 3 in a basic aqueous bath in order to obtain the composition in gel form, and
5) optionally washing, drying and/or sterilizing the gel obtained in step 4, preferably by autoclave.

### BRIEF DESCRIPTION OF THE DRAWINGS

Other features, details and advantages will become apparent upon reading the following detailed description and upon examining the appended drawings, in which:

FIG. 1 shows a photo of the hydrogels in disk form according to example 4; top left: composition 9 according to the invention (HG-5-5); bottom left: composition 12 according to the invention (HG-8-8); bottom right: composition 10 according to the invention (HG-5-10); top right: composition 11 according to the invention (HG-5-15).

FIG. 2 shows a photo of the hydrogels in cylinder form obtained according to example 4. This is comparative composition 2 (Ref-HG-8-0).

FIG. 3 shows a photo of the hydrogels in cylinder form obtained according to example 4. This is composition 10 according to the invention (HG-5-10).

FIG. 4 shows an optical micrograph of a thread having a knot according to example 5.

FIG. 5 shows an optical micrograph of two braided threads according to example 5.

FIG. 6 shows a photo of the dried gel according to example 6.

FIG. 7 shows a photo of the swollen gel according to example 6.

FIG. 8 shows an optical micrograph of a thread, half of which has been submerged in water, according to example 6.

FIG. 9 shows an optical micrograph of a thread according to example 6, onto just a portion of which a droplet of water has been deposited.

FIG. 10 shows a photo of the formation of a gel according to example 12 after injection of composition 23 according to the invention (IS-1,7-3,3) in PBS at 10 mM.

FIG. 11 shows MRI images (7.1 T) of a gel formed just after injection (day 0) and after 15 days and 30 days according to example 14.

FIG. 12 shows MRI images (7.1 T) of the gel formed just after injection according to example 14.

### DEFINITIONS

In the present disclosure, all the percentages and ppm are indicated by weight, unless otherwise specified.

Unless otherwise indicated, all the viscosities referred to in the present disclosure correspond to a “Newtonian” dynamic viscosity at 25° C., i.e. the dynamic viscosity which is measured in a manner known per se with a rheometer having a shear rate gradient which is sufficiently low for the viscosity measured to be independent of the rate gradient.

For the purpose of the present disclosure, “chitosan” means a natural copolysaccharide-type polymer consisting of a random distribution (statistical copolymer) or non-random distribution (block or sequenced copolymers) of D-glucosamine (GlcN) and of N-acetyl-D-glucosamine (GlcNAc), or even exclusively of D-glucosamine, bonded by glycosidic linkages of p(1->4) type. Chitosan is not particularly present in the native state in biomass, and is mainly obtained by chemical modification of chitin, of which it is a derivative. Chitin has a structural role; it is mainly found in some fungi in which it forms the cell wall (Basidiomycetes, for example: Agariscus campestris, Agariscus bisporus, Ascomycetes, Zygomycetes, and Deuteromycetes), but it also forms the exoskeleton of arthropods (crustaceans and insects), particularly in prawns and crabs, and the endoskeleton of cephalopods such as squid or cuttlefish. Chitin is converted to chitosan by deacetylation, i.e. by the alkaline hydrolysis of acetyl groups to generate primary amine groups. Chitosan is a biodegradable and biocompatible polymer having bacteriostatic and fungistatic properties.

“Gel” means a non-fluid polymer network which is swollen by a solvent. The polymer network is a network consisting of crosslinked polymer chains. The interactions responsible for crosslinking the polymers can be physical or chemical. Advantageously, in the context of the invention, the gels consist exclusively of chitosan A, of copolysaccharide B, and optionally of water and/or an active pharmaceutical ingredient.

“Hydrogel” means a viscoelastic material comprising at least 60% by weight of water, and preferably at least 80% by weight of water. The hydrogel according to the invention generally contains from 0.1% to 40% and preferably from 0.5% to 20% by weight of the mixture of chitosan A and copolysaccharide B.

In the context of the invention, the hydrogel is said to be physical because the interactions responsible for the inter-chain crosslinking that gives the hydrogel its cohesion are physical, and are in particular hydrogen bonds and/or hydrophobic interactions, as opposed to a “chemical” hydrogel (also referred to as a crosslinked hydrogel) in which the inter-chain interactions are of the covalent bond type. No chemical crosslinker is present in a purely physical hydrogel. Advantageously, in the context of the invention, the physical hydrogels consist exclusively of water, of chitosan A, of copolysaccharide B, and optionally of a pharmaceutically active ingredient, and preferably contain more than 80% (w/w) of water. In particular, such hydrogels do not comprise collagen, polycaprolactone, or toxic chemical crosslinkers (of the glutaraldehyde, formaldehyde, epichlorohydrin type, etc.). Having a physical hydrogel has advantages, because chemical hydrogels are poorly resorbable due to the stability of the covalent bonds, whereas the physical hydrogel of chitosan A and of copolysaccharide B according to the present invention is resorbable in physiological medium, in particular under mildly acid conditions.

“Xerogel” means a material obtained by drying, in particular drying of a hydrogel, comprising less than 60% by weight of water, preferably less than 50% by weight of water and more preferentially less than 20% of water. The xerogel according to the invention generally contains at least 40% by weight of the mixture of chitosan A and of copolysaccharide B, preferably between 40 and 100%, preferentially between 50 and 99.9% of the mixture, and more preferentially between 80 and 99.5% of the mixture.

“Aerogel” means a material which is structurally similar to a hydrogel but in which the water has been replaced by gas using a process that prevents the impact of the capillary forces of the solvent on the material (cf. Mike Robitzer, Laurent David, Cyrille Rochas, Francesco Di Renzo and Françoise Quignard, Nanostructure of calcium alginate aerogels obtained from multistep solvent exchange route, Langmuir 2008, 24, 12547-12552, and Mike Robitzer, Laurent David, Cyrille Rochas, Francesco Di Renzo and Frangoise Quignard, Supercritically-dried alginate aerogels retain the fibrillar structure of the hydrogels, Macromol. Symp. 2008, 273, 80-84).

In the context of the invention, the weight-average molar masses Mw of the chitosan A and of the copolysaccharide B are determined by size exclusion chromatography, the experimental conditions of which are described in the publication “Physico-chemical studies of the gelation of chitosan in a hydroalcoholic medium”, A. MONTEMBAULT, C. VITON, A. DOMARD, Biomaterials, 26(8), 933-943, 2005.

The degree of acetylation (DA) of the chitosan A and of the copolysaccharide B is determined using the proton NMR technique, following Hirai's methodology (A. HIRAI, H ODANI, A. NAKAJIMA, Polymer Bulletin, 26 (1), 87-94, 1991).

In the context of the invention, the degree of crystallinity represents the proportion of material in the crystalline state. For polysaccharides, it is often determined by X-ray diffraction (Alexander, L. E., X-ray Diffraction Methods in Polymer Science, Wiley-Interscience, New York, 1969, p. 137) because many polysaccharides degrade at temperatures below the melting point of the crystalline phase, which does not make it possible to use differential scanning calorimetry. It is therefore this method which is to be considered in the context of the invention. Other spectroscopic methods are possible, but have to be adapted in each case (Fourier-transform infrared spectroscopy, Raman spectroscopy). In principle, determining the density by a gradient column makes it possible to calculate the degree of crystallinity, having knowledge of the density of the amorphous phase and of the crystalline phase of the polymer, but many polysaccharides can swell and absorb the solvents used for density gradient columns, thus further limiting the use of this method.

In the context of the invention, the size of the crystallites can be determined by studying the width of the diffraction peaks obtained by the powder method, using the Scherrer equation (Alexander, L E., ‘X-ray Diffraction Methods in Polymer Science’, Wiley-Interscience, New York, 1969, p. 137).

### DESCRIPTION OF THE EMBODIMENTS

Firstly, the invention relates to a composition comprising:
a. at least one chitosan A, and
b. at least one statistical copolysaccharide B having a weight-average molecular weight of between 100 kDa and 1000 kDa, of formula I:

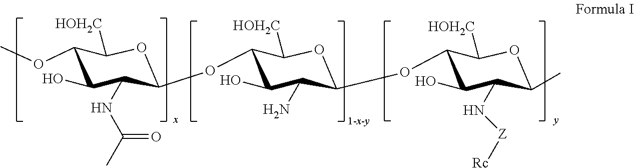

in which:
each Rc independently represents a group comprising a chelating agent,
each Z independently represents a linker which may be a single bond or a hydrocarbon-based chain comprising between 1 and 12 carbon atoms, it being possible for said chain to be linear or branched and to comprise one or more unsaturations and to comprise one or more heteroatoms, preferably selected from nitrogen, oxygen, sulfur and atoms of the halogen family,
x is between 0.01 and 0.5.
y is between 0.05 and 0.5,
the y/x ratio being greater than 0.2, preferably greater than 1,
the sum x+y being greater than 0.1, and
less than 10% of groups comprising a chelating agent of Rc type which are chelated by a cation of a transition element selected from block d and f elements, and
c. optionally water.

According to one embodiment, the weight ratio between A and B (A:B) is between 2:1 and 1:10, preferably between 1:1 and 1:5.

This composition can additionally comprise at least one active pharmaceutical ingredient, preferably selected from anticancer agents, antibacterials, antifungals, anti-inflammatories, messenger RNAs, proteins and antigens.

Active pharmaceutical ingredient means any compound that has a therapeutic or preventative effect. The fact that this composition has good swelling properties makes it possible to effectively capture then release active pharmaceutical ingredients. Indeed, it is possible to bring the composition into contact with the active pharmaceutical ingredient, for example with a solution comprising the active pharmaceutical ingredient, and the composition then captures this ingredient. This ingredient can subsequently be released, for example by submerging the composition in another medium. This advantageously takes place when the composition is in gel form.

The composition may be in a plurality of forms: aqueous solution, lyophilizate or gel.

According to one embodiment, the composition is an aqueous solution, preferably comprising at least 10 g/l, preferably at least 50 g/l, of the mixture of chitosan A and of copolysaccharide B. When the composition is in the form of an aqueous solution, the composition may be injectable. The concentration of the mixture of chitosan A and of copolysaccharide B may be between 10 g/l and 500 g/l, preferably between 40 g/l and 160 g/l.

The aqueous solution may have a Newtonian viscosity of between 0.1 and 50 000 Pa·s, preferably of between 50 and 25 000 Pa·s, and more preferentially of between 100 and 10 000 Pa·s.

The aqueous solution is a gellable solution. It can gel when it is brought into contact with a coagulation bath at basic pH. The gelling takes place at basic pH after neutralization of the amines (NH₂) which were initially protonated (NH₃⁺) present in the solution. The entangled state of the chains resulting from the high initial viscosity is fixed by the formation of inter-chain interaction sites (crystallites, hydrophobic interactions, hydrogen bond interactions) and thereby provides a stable physical hydrogel having adequate mechanical properties to be handled or stretched. The resulting hydrogels are reversible by treatment in acid solution, and differ from chemical hydrogels in which the gelling is provided by the formation of irreversible covalent chemical bonds. They can be reversibly dried and rehydrated. The solution can also be lyophilized.

The composition can also be in the form of a lyophilizate or a gel, preferably in the form of a hydrogel, a xerogel or an aerogel.

The hydrogel according to the invention can contain from 0.1% to 40% and preferably from 0.5% to 20% by weight of the mixture of chitosan A and copolysaccharide B.

The xerogel according to the invention can contain at least 40% by weight of the mixture of chitosan A and of copolysaccharide B, preferably between 40 and 100%, preferentially between 50 and 99.9% of the mixture, and more preferentially between 80 and 99.5% of the mixture. When the composition is in the form of a xerogel, the xerogel can result from the at least partial drying of a hydrogel.

The xerogel, when it is in contact with an aqueous medium or biological tissue for a period of at least 1 hour, swells to form a hydrogel, said hydrogel having a volume at least twice as large, preferably at least 5 times as large, as the volume of reference xerogel before contact with the aqueous medium or biological tissue. The swelling can be quantified by measuring the weight of the initial xerogel and the weight of the hydrogel obtained.

A biological tissue is a set of differentiated or undifferentiated cells organized in a characteristic architecture, in combination with a network of natural macromolecules or extracellular matrix (ECM), all contributing to performing the same function. Cellular tissue is naturally hydrated, allowing the xerogel to swell.

The hydrogel obtained by rehydration of the xerogel can subsequently be dried again in order to re-form a xerogel. It is possible to perform a plurality of drying-swelling cycles with this composition.

The gels according to the invention have good mechanical properties. It is thus possible to make threads therefrom, for example by extruding the composition according to the invention through a die in a coagulation bath. These threads can subsequently be used to prepare a textile material. The thread can have a diameter of between 50 μm and 700 μm, preferably between 80 μm and 500 μm.

The composition according to the invention, and in particular the threads and/or textile materials, can be used in medical devices by virtue of their good properties of swelling and capturing metals. Advantageously, the threads and/or textile materials can be degraded by the body or can be explanted in order to eliminate the chelated metals.

The medical device in question is preferably a dressing, an implant, or a dermal filler product. According to one embodiment, the medical device comprises a thread and/or a textile material according to the invention.

In the case of a dermal filler product, said product can be in the form of a thread, said thread preferably being in the form of a xerogel which can swell on contact with an aqueous medium or a biological tissue in order to form a hydrogel. The dermal filler product can also be a solution according to the invention, which can then be injected.

The good metal capturing properties of the composition make it possible to use it to capture at least one metal in the treatment or prevention of endometriosis, fungal infections, bacterial infections, chronic wounds (pressure sores, diabetic wounds), neurodegenerative disorders (Parkinson's disease, Alzheimer's disease), hemochromatosis, Wilson's disease or lead poisoning. The metal is preferably a metal cation. According to one embodiment, the metal belongs to the group consisting of copper, iron, lead, zinc, aluminum, gadolinium and manganese, and more preferentially the group consisting of copper, iron and lead.

The invention also relates to the use of the composition according to the invention for capturing at least one metal, optionally for treating or preventing a disease.

The invention also relates to a method for treating or preventing endometriosis, fungal infections, bacterial infections, chronic wounds (pressure sores, diabetic wounds), neurodegenerative disorders (Parkinson's disease, Alzheimer's disease), hemochromatosis, Wilson's disease or lead poisoning, said method comprising the use of the composition according to the invention for capturing at least one metal.

### Method for Preparing the Composition

The invention also relates to a method for preparing a composition according to the invention, comprising the following steps:
1) dispersing the chitosan A and the statistical copolysaccharide B having a weight-average molecular weight of between 100 kDa and 1000 kDa of formula I in water,
2) adding an acid, preferably acetic acid,
3) mixing then centrifuging the solution obtained in order to recover a solution comprising the composition,
4) optionally treating the solution obtained in step 3 in a basic aqueous bath in order to obtain the composition in gel form, and
5) optionally washing, drying and/or sterilizing the gel obtained in step 4, preferably by autoclave.

Preferably, the acid is added in stoichiometric proportion relative to the non-functionalized functions of primary amine type of the chitosan A and of the copolysaccharide B. The acid is preferably organic.

The solution comprising the composition preferably has a viscosity of between 0.1 and 50 000 Pa·s.

The basic aqueous bath in step 4 is a coagulation bath which enables the gelling of the solution. This coagulation bath is preferably an alkaline solution, for example sodium hydroxide, aqueous ammonia or potassium hydroxide, at a concentration which can be between 0.5 and 10 M, preferably between 1 and 5 M. The coagulation bath can also be a coagulation chamber in which alkaline vapors are used, such as ammonia vapor.

To obtain a thread, it is possible, during step 4, to extrude the composition according to the invention through a die (or extrusion cone). The extrudate is then introduced into the coagulation bath. It is also possible to directly extrude the composition into the coagulation bath.

Step 5 can comprise one or more washing steps, it being possible for the washing to be carried out using water or with a buffer.

Step 5 can also comprise one or more solvent exchange steps, for example to replace the water with ethanol.

Step 5 can comprise one or more drying steps. The drying in step 5 can be carried out in the open air, at ambient temperature, or at a temperature of between 30 and 250° C., for example under hot air at a temperature of between 100 and 200° C. According to one embodiment, the drying is carried out after exchanging water for ethanol using consecutive baths which are gradually more concentrated in alcohol, then after exchanging ethanol for liquid CO₂ in a pressurized chamber, then after expansion in a supercritical CO₂ medium, which makes it possible to form an aerogel.

Any sterilization technique well known to those skilled in the art can be used, in particular steam sterilization by autoclave, or gamma or beta radiation.

### Chitosan A

The composition according to the invention comprises a chitosan A.

The composition can have:
a degree of crystallinity, in relation to the chitosan A, of at least 10% relative to the total dry weight of the composition, and
a crystallite size of the chitosan A of less than 20 nm.

The degree of crystallinity in relation to the chitosan A can be between 10 and 25%. The crystallites of the chitosan A serve as physical crosslinking nodes which are essential for forming a gel. The size of the crystallites can be for example between 1 and 20 nm.

Advantageously, the chitosan A has an average molecular weight Mw of between 100 kg/mol and 1000 kg/mol, preferably between 200 kg/mol and 700 kg/mol.

According to a preferred embodiment, the chitosan A has a degree of acetylation x of less than 40%, preferably less than 10%, for example of between 0% and 10%.

### Capolysaccharide B

The composition comprises a statistical copolysaccharide B having a weight-average molecular weight of between 100 kDa and 1000 kDa, of formula 1.

It is understood that, in the above formula I, a plurality of groups Rc can be present in the polysaccharide. These groups Rc can be identical to or different from one another. They are all independently selected from groups bearing a chelating agent. The same applies to the linkers Z: a plurality of linkers Z can be present, and they can be identical to or different from one another.

The copolysaccharide B can be of formula II:

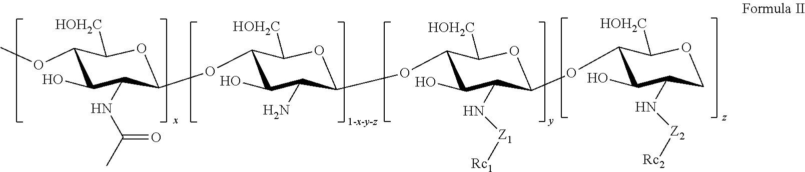

in which:
Rc₁ and Rc₂ are different and are groups comprising a chelating agent,
Z₁ and Z₂, which are identical or different, are linkers which may be a single bond or a hydrocarbon-based chain comprising between 1 and 12 carbon atoms, it being possible for said chain to be linear or branched and to comprise one or more unsaturations and to comprise one or more heteroatoms, preferably selected from nitrogen, oxygen, sulfur and atoms of the halogen family,
x is between 0.01 and 0.5, preferably between 0.01 and 0.1, and preferentially between 0.05 and 0.1,
y is between 0.01 and 0.5, preferably between 0.05 and 0.2,
z is between 0 and 0.2,
and
less than 10% of groups comprising a chelating agent of Rc1 and Rc2 type which are chelated by a cation of a transition element selected from block d and f elements.

Group of Rc type means the groups Rc in the polysaccharide of formula I, and the groups Rc₁ and Rc₂, when the group Rc₂ is present, in the polysaccharide of formula II.

According to one embodiment, less than 10% of the groups of Rc type, preferably less than 5%, are chelated by a cation, in particular a metal cation. The fact that the groups of Re type are in a free form enables good metal capture. The copolysaccharide B is also highly hydrophilic, causing good swelling properties.

In accordance with the invention, the groups Rc, Rc₁ and Rc₂ are chelating agents. In other words, the groups Rc, Rc₁ and Rc₂ make it possible to chelate one or more metals by forming a complex.

Each of the groups Rc, Rc₁ and Rc₂ can contain one or more coordination sites. The coordination site is preferably a nitrogen or oxygen atom. Advantageously, each of the groups Rc, Rc₁ and Rc₂ comprises between 4 and 8 coordination sites, more advantageously between 6 and 8 coordination sites, and even more advantageously each of the groups Rc, Rc₁ and Rc₂ comprises 8 coordination sites.

A coordination site means a single function capable of bonding to a metal. For example, an amine function represents a coordination site by the formation of a dative bond between the nitrogen atom and the metal, and a hydroxamic acid function also represents a coordination site by the formation of a dative bond between the oxygen of the carbonyl unit and by a covalent bond with the oxygen of the N-oxide unit, the coordination site thereby forming a five-membered ring.

In one embodiment of the invention, for the polysaccharide of formula I, each group Rc is independently selected from the group consisting of DOTA (N,N′,N″N′″-(1,4,7,10-tetraazacyclododecane)tetraacetic acid), NOTA (1,4,7-triazacyclononane-1,4,7-triacetic acid), NODAGA (1,4,7-triazacyclononane-1-glutaric acid-4,7-diacetic acid), DOTAGA (2-(4,7,10-tris(carboxymethyl)-1,4,7,10-tetraazacyclododecan-1-yl)pentanedioic acid), DOTAM (1,4,7,10-tetrakis(carbamoylmethyl)-1,4,7,10-tetraazacyclododecane), NOTAM (1,4,7-tetrakis(carbamoylmethyl)-1,4,7-triazacyclononane), DOTP (1,4,7,10-tetraazacyclododecane-1,4,7,10-tetrakis(methylene phosphonate), NOTP (1,4,7-tetrakis(methylene phosphonate)-1,4,7-triazacyclononane), TETA (1,4,8,11-tetraazacyclotetradecane-N,N′,N″, N″′-tetraacetic acid), TETAM (1,4,8,11-tetraazacyclotetradecane-N,N′, N″, N″′-tetrakis(carbamoyl methyl), DTPA (diethylenetriaminepentaacetic acid) and DFO (deferoxamine), preferably from the group consisting of DOTAGA, DFO, DOTAM and DTPA, and the group Rc is more preferably DOTAGA.

In one embodiment of the invention, for the polysaccharide of formula II, Rc₁ and Rc₂ are independently selected from the group consisting of DOTA, NOTA, NODAGA, DOTAGA, DOTAM, NOTAM, DOTP, NOTP, TETA, TETAM, DTPA and DFO, preferably from the group consisting of DOTAGA, DFO, DOTAM and DTPA.

According to one embodiment, for the polysaccharide of formula II, the group Rc₁ is DOTAGA, and preferably z=0.

According to one embodiment, for the polysaccharide of formula II, the group Rc₁ is DOTAGA, and the group Rc₂ is DFO.

Linker of Z type means the linkers Z in the polysaccharide of formula I, and the linkers Z₁ and Z₂, when the linker Z₂ is present, in the polysaccharide of formula II.

The choice of the linkers Z, Z₁ and Z₂ in formulas I and ∥ essentially depends on the groups Rc, Rc₁ and Rc₂, and on the metal to be chelated. This is because, particularly for steric reasons, the groups Rc, Rc₁ and R_{c} can be closer to or further from the nitrogen of the 6-membered ring of the glucosamine unit.

Preferably, in formula I, each Z is independently a single bond or a hydrocarbon-based chain comprising between 1 and 12 carbon atoms, it being possible for said chain to be linear or branched and to comprise one or more unsaturations and to comprise one or more heteroatoms, preferably selected from nitrogen, oxygen, sulfur and atoms of the halogen family.

According to one embodiment, in formula 1, each Z is independently selected from the group consisting of: a bond, a linear or branched alkyl chain comprising between 1 and 12 carbon atoms, and a linear or branched alkenyl chain comprising between 2 and 12 carbon atoms, it being possible for said alkyl and alkenyl chains to be interrupted by one or more C₆-C₁₀ aryl groups and/or by one or more heteroatoms or groups selected from the group consisting of —O—, —S—, —C(O)—, —NR′—, —C(O)NR′—, —NR′—C(O)—, —NR′—C(O)—NR′—, —NR′—C(O)—O—, —O—C(O)NR′, —C(S)NR′—, —NR—C(S)—, —NR′—C(S)—NR′,
it being possible for said alkyl and alkenyl chains to be substituted by one or more groups selected from the group consisting of halogens, —OR′, —COOR′, —SR′, —NR′₂,
each R′ is independently H or a C₁-C₆ alkyl.

Advantageously, in formula I, each Z is independently selected from the group consisting of: a bond and a linear or branched alkyl chain comprising between 1 and 12 carbon atoms, it being possible for said alkyl chain to be interrupted by one or more C₆-C₁₀ aryl groups and/or by one or more heteroatoms or groups selected from the group consisting of —O—, —S—, —C(O)—, —NR′—, —C(O)NR′—, —NR′—C(O)—, —C(S)NR′—, —NR′—C(S)—, —NR′—C(S)—NR′, each R′ is independently H or a C₁-C₆ alkyl.

In a particular embodiment, each Z is an alkyl chain comprising between 1 and 12 carbon atoms.

In another particular embodiment, each Z is a polyethylene glycol (PEG) segment.

Preferably, in formula II, Z₁ and Z₂ are independently a single bond or a hydrocarbon-based chain comprising between 1 and 12 carbon atoms, it being possible for said chain to be linear or branched and to comprise one or more unsaturations and to comprise one or more heteroatoms, preferably selected from nitrogen, oxygen, sulfur and atoms of the halogen family.

According to one embodiment, in formula II, Z₁ and Z₂ are independently selected from the group consisting of: a bond, a linear or branched alkyl chain comprising between 1 and 12 carbon atoms, and a linear or branched alkenyl chain comprising between 2 and 12 carbon atoms, it being possible for said alkyl and alkenyl chains to be interrupted by one or more C₆-C₁₀ aryl groups and/or by one or more heteroatoms or groups selected from the group consisting of —O—, —S—, —C(O)—, —NR′—, —C(O)NR′—, —NR—C(O)—, —NR′—C(O)—NR′—, —NP—C(O)—O—, —O—C(O)NR′, —C(S)NR′—, —NR′—C(S), —NR—C(S)—NR′,
it being possible for said alkyl and alkenyl chains to be substituted by one or more groups selected from the group consisting of halogens, —OR′, —COOR, —SR′, —NR′₂,
each R′ is independently H or a C₁-C₆ alkyl.

Advantageously, in formula II, Z₁ and Z₂ are independently selected from the group consisting of: a bond and a linear or branched alkyl chain comprising between 1 and 12 carbon atoms, it being possible for said alkyl chain to be interrupted by one or more C₆-C₁₀ aryl groups and/or by one or more heteroatoms or groups selected from the group consisting of —O—, —S—, —C(O)—, —NR′—, —C(O)NR′—, —NR′—C(O)—, —C(S)NR′—, —NR′—C(S)—, —NR′—C(S)—NR′, each R′ is independently H or a C₁-C₆ alkyl.

In a particular embodiment, Z₁ and/or Z₂ is an alkyl chain comprising between 1 and 12 carbon atoms.

In another particular embodiment. Z₁ and/or Z₂ is a polyethylene glycol (PEG).

In accordance with the invention, z is between 0 and 0.2. In other words, the units of type C can be exclusively units comprising Z₁ as linker and Rc₁ as group bearing a chelating agent.

The polysaccharide according to the invention has a weight-average molecular weight of between 100 kDa and 1000 kDa; advantageously, the weight-average molecular weight of the polysaccharide according to the invention is between 200 kDa and 750 kDa, more advantageously between 250 kDa and 500 kDa, and even more advantageously between 300 kDa and 400 kDa.

According to one embodiment, the copolysaccharide B is selected from the following polysaccharides:
copolysaccharide of formula II in which y=0.15, Rc₁ is DOTAGA and Z₁ is a bond;
copolysaccharide of formula II in which 0.05≤z<0.06, Rc₁ is DOTAGA and Z₁ is a bond, and Rc₂ is DFO and Z₂ is selected from the group consisting of: a bond and a linear or branched alkyl chain comprising between 1 and 12 carbon atoms,
it being possible for said alkyl chain to be interrupted by one or more C₆-C₁₀ aryl groups and/or by one or more heteroatoms or groups selected from the group consisting of —O—, —S—, —C(O)—, —NR′—, —C(O)NR′—, —NR′—C(O)—, —C(S)NR′—, —NR′—C(S)—, —NR′—C(S)—NR′, each R′ is independently H or a C₁-C₆ alkyl.

The copolysaccharide B can be obtained according to a method comprising the following three consecutive steps:
Step 1: dissolving a chitosan in an acid solution at a pH of between 4 and 5;
Step 2: partial acetylation of the amine functions of the chitosan dissolved in step 1 (formation of acetylated units);
Step 3: functionalization of at least some of the amine functions still present at the end of step 2 (formation of units comprising the group Rc).

Step 3 can be subdivided into a plurality of steps, particularly when the linker Z is a hydrocarbon-based chain as defined above.

In the embodiments in which the linker Z is a hydrocarbon-based chain as defined above, step 3 can comprise a sub-step 3-1 which consists in grafting said hydrocarbon-based chain onto at least some of the amine functions still present at the end of step 2, then a sub-step 3-2 which consists in grafting the group Rc onto said hydrocarbon-based chain. Alternatively, step 3 does not comprise sub-steps. In this alternative, said hydrocarbon-based chain is coupled with the group Rc prior to step 3; said step 3 is thus carried out with a molecule comprising the group Rc and said hydrocarbon-based chain.

Alternatively, the copolysaccharide B can be obtained from a chitosan having the desired degree of acetylation; thus, in this embodiment, the acetylated units are already present and do not need to be formed. In this embodiment, the method for obtaining the copolysaccharide B comprises at least the following two consecutive steps:
Step 1b: dissolving a partially acetylated chitosan in an acid solution at a pH of between 4 and 5;
Step 2b: functionalization of at least some of the amine functions of said partially acetylated chitosan dissolved in step 1b (formation of units comprising the group Rc).

In the same way as above for said step 3, said step 2b can be subdivided into a plurality of steps, particularly when the linker Z is a hydrocarbon-based chain as defined above.

In the embodiments in which the linker Z is a hydrocarbon-based chain as defined above, step 2b can comprise a sub-step 2b-1 which consists in bonding said hydrocarbon-based chain to at least some of the amine functions, then a sub-step 2b-2 which consists in grafting the group Rc onto said hydrocarbon-based chain. Alternatively, step 2b does not comprise sub-steps. In this alternative, said hydrocarbon-based chain is coupled with the group Rc prior to step 2b; said step 2b is thus carried out with a molecule comprising the group Rc and said hydrocarbon-based chain.

### EXAMPLES

### Materials and Methods

A plurality of chitosans A and copolysaccharides B were used to prepare the compositions according to the invention. Table 1 summarizes the compounds used. The syntheses of compounds B1 and B2 are presented in examples 1 and 2, respectively.

### [Table 1]

TABLE 1
List of compounds used and their properties

| | | Average | (DA) | (DS) |
|---|---|---|---|---|
| | | molecular | Degree of | Degree of |
| | | weight | acetylation | substitution |
| Products | Name | (kDa) | (%) | (%) |
| A1 - Chitosan having | Chitosan | 250 | 6.5 | 0 |
| intermediate molecular | 250 | | | |
| weight | | | | |
| A2 - Chitosan having | Chitosan | 650 | 6.7 | 0 |
| high molecular weight | 650 | | | |
| B1 - N-DOTAGA | MEX-CD2 | 250 | 6.5 | 13-16 |
| chitosan having low | | | | |
| DA and intermediate | | | | |
| molecular weight | | | | |
| B2 - N-DOTAGA, DFO | MEX- | 250 | 26 | 8.6% |
| acetylated chitosan | | | | DOTAGA |
| having intermediate | CDDFO1 | | | 0.7% DFO |
| molecular weight | | | | |
| B3 - N-DFO | MEX- | 250 | 41 | 2.9 |
| Chitosan having | CDFO | | | |
| intermediate molecular | | | | |
| weight | | | | |

The degree of acetylation (DA) of compounds B1 and B2 is obtained as follows: The 1H NMR spectra of the products to be analyzed are obtained using an Avance III HD 400 MHz NanoBay spectrometer from Bruker. The phase is corrected manually using the water peak, located at 4.7 ppm. The peaks are integrated manually by integrating the set of peaks between 4.1 and 2.9 ppm and by integrating the peak to 2.00 ppm between 2.02 and 1.90 ppm. The DA as a percentage is directly obtained by normalizing the integration of the set of peaks to 200.

The degree of substitution with DOTAGA (DS) is obtained as follows: different samples of the product to be analyzed are obtained by redispersing the lyophilized product in an acetate buffer (0.1 M acetic acid and 0.1 M ammonium acetate) to obtain a final polymer concentration by weight of 0.1%. Different volumes of a copper nitrate solution are added to obtain copper concentrations in each sample ranging from 0 to 1 mM, then each sample is stirred. The absorbance of the resulting solutions is subsequently measured using a Varian Cary® 50 UV-visible spectrophotometer. The DS is determined by plotting the absorbance at 295 nm (absorption maximum for DOTAGA) as a function of the copper concentration, with the discontinuity region corresponding to the amount of DOTAGA per 1 g of product.

The purity of compounds B1 and B2 is verified as follows: the HPLC-SEC-UV chromatograms of the products to be analyzed were recorded on samples at a 1% concentration by weight of polymer using a Shimadzu Prominence HPLC system. The SEC column used is a PolySep-GFC-P 4000 column and an acetate buffer is used as eluent (0.1 M acetic acid and 0.1 M ammonium acetate). The working temperature is 30° C. and the absorption wavelength is 295 nm. The eluent flow rate is 0.8 ml/min. The purity of the product is verified by integration the peak of free DOTAGA over the integration of the peak of chitosan grafted with DOTAGA.

### Example 1—Synthesis of MEX-CD2 (B1)

The chitosan precursor of the polysaccharide MEX-CD2 (B1) is medical grade and of animal origin. The weight-average and number-average molar masses (M_{w}=2.583×10⁵ g/mol, Mₙ=1.323×10⁵ g/mol, respectively) were determined by size exclusion chromatography coupled with refractive index and multiangle laser light scattering measurements. The degree of acetylation (proportion of N-acetyl-D-glucosamine units) of such a chitosan was determined by ¹H NMR spectroscopy using Hirai's method (Asako Hirai et al., Determination of degree of deacetylation of chitosan by 1H NMR spectroscopy, Polymer Bulletin, 1991, 26, 87-94) and is estimated to be 6±05%.

60 g of chitosan are introduced into a 10 l reactor with 4 l of ultrapure water and 50 ml of acetic acid, then the mixture is placed under mechanical stirring at 500 rpm. After complete dissolution of the chitosan (3 h), 4 l of 1,2-propanediol are added to the medium and the mixture is kept under stirring until homogenized (2 h). 120 g of DOTAGA anhydride are subsequently introduced and the mixture is kept under stirring overnight until completely dissolved. The synthesis product is subsequently purified by tangential flow filtration using the Sartoflow® Advanced device with a Sartocon® Slice PESU cassette (polyethersulfone membranes; cutoff threshold: 100 kDa; filtration surface area: 0.1 m²) according to a diafiltration-concentration model against 200 l of a 0.1 M acetic acid solution then 200 l of a 5 mM acetic acid solution. The purification is monitored by size exclusion chromatography coupled to a UV detector, until less than 5% free DOTAGA is obtained. The product is subsequently lyophilized and the degree of acetylation (DA) and the degree of substitution (DS) are determined by ¹H NMR and the copper chelation method described above, respectively. The purity is verified by HPLC as mentioned above.

### Example 2—Synthesis of MEX-CDDFO1 (B2)

The chitosan precursor of the polysaccharide MEX-CDDFO1 (B2) is identical to that described in example 1 (M_{w}=2.583×10⁵ g/mol, Mₙ=1.323×10⁵ g/mol, DA=6±0.5%).

60 g of chitosan, 4 l of ultrapure water and 45 ml of glacial acetic acid are introduced into a 10 l reactor and placed under stirring for a duration of 16 h at a pH of 4.5±0.5. 1.2 l of propane-1,2-diol are added to the solution and stirring is maintained for 1 h. A solution composed of 14 ml of acetic anhydride dissolved in 600 ml of propane-1,2-diol is subsequently added slowly over 30 min, the reaction medium is kept under stirring for 4 h. 120 g of DOTAGA anhydride are subsequently weighed out and added to the reactor, then 2 l of propane—1.2-diol are added and stirring is maintained for 16 h, At the end of the reaction, the solution is purified by tangential filtration using a 100 kDa membrane. The synthesis product is subsequently purified by tangential filtration in the same way as described in example 1, against 200 l of a 0.1 M acetic acid solution then 200 l of ultrapure water. The purification is monitored by size exclusion chromatography coupled to a UV detector, until less than 5% free DOTAGA is obtained. The product is subsequently lyophilized at a concentration of 7 g/l and the degree of acetylation (DA) and the degree of substitution (DS) are determined by ¹H NMR and the copper chelation method described above, respectively.

A volume of 720 ml of purified chitosan-DOTAGA at a concentration of 7 g/l is introduced into a 2 l round-bottomed flask. This solution (pH between 6 and 6.5) is supplemented with ultrapure water to reach a total volume of 900 ml. In parallel, 143.1 mg of p-NCS-Bz-DFO are weighed out and dissolved in 100 ml of DMSO. This solution is subsequently added dropwise to the chitosan-DOTAGA solution. The solution is kept under stirring and heated at a temperature of 40° C. overnight. 500 ml of this solution are diluted to 5 l with ultrapure water then reconcentrated to 1 l by tangential filtration using the Sartoflow® Smart device with two Sartocon® Slice PESU cassettes (polyethersulfone membranes; cutoff threshold: 100 kDa; filtration surface area: 0.02 m²). The filtration is continued at a constant concentration against 4 l of ultrapure water, then the solution is reconcentrated to 500 ml.

HPLC-SEC-UV analysis of the product makes it possible to confirm the grafting of the DFO and the elimination of the residual p-NCS-Bz-DFO. Comparison of the HPLC-SEC-UV of the chitosan-DOTAGA and of the chitosan-DOTAGA-DFO shows an increase in the absorption of the polymer peak (around 7 min) when the p-NCS-Bz-DFO is grafted.

In the case of the MEX-CDDFO1 (B2), the copper chelation method was applied to the product before functionalization with DFO, and made it possible to determine a DOTAGA DS of 8.6%. A method analogous to the copper chelation method made it possible to determine the DFO DS. Increasing iron(III) concentrations are added to a 0.1 g/l solution of MEX-CDDFO1 (B2) in acetate buffer at pH 4.5 (0.1 M ammonium acetate and 0.1 M acetic acid). The absorption measured at 425 nm (λmax of the [Fe(η⁶-DFO)] complex) is subsequently plotted as a function of the iron concentration, and the iron concentration corresponding to the discontinuity region of the gradient makes it possible to obtain the DS. This method makes it possible to estimate the DS of MEX-CDDFO1 (B2) at 0.7%.

¹H NMR analysis of the intermediate product (N-DOTAGA chitosan) according to the method described above made it possible to determine a DA of 26%. ¹H NMR analysis of MEX-CDDFO1 (B2) reveals the presence of a peak at 7.3 ppm, characteristic of the protons of a substituted benzene. The NMR spectrum of the product also has peaks between 1.1 and 1.7 ppm which are identical to those of deferoxamine mesylate alone, confirming the effective grafting of the DFO to the N-DOTAGA chitosan after purification.

### Example 3—Synthesis of MEX-CDFO (B3)

The chitosan precursor of the polysaccharide MEX-CDFO is identical to that described in example 1 (Mw=2.583×10⁵ g/mol, Mn=1.323×10⁵ g/mol, DA=6±0.5%). For 2.5 g of chitosan, 170 ml of ultrapure water and 2.25 ml of glacial acetic acid are introduced into a 1 l reactor and placed under stirring until the chitosan has completely dissolved. 50 ml of propane-1,2-diol are added to the solution. A solution composed of 785 μl of acetic anhydride dissolved in 25 ml of propane-1,2-diol is subsequently added slowly, the reaction mixture is kept under stirring for 4 hours. 40 ml of ultrapure water and 43 ml of a 1 M solution of NaOH are added in order to obtain a solution between pH 5.5 and 6.5. A volume of 75 ml of propane—1,2-diol and 35 ml of DMSO are added. Simultaneously, 576 mg of p-NCS-Bz-DFO are weighed out and dissolved in 57.6 ml of DMSO. This solution is added at a rate of 150 μl/min. During the addition of the DFO, 3.1 ml of 1 M HCl are also added at a rate of 8 μl/min. The solution is kept under stirring and heated at a temperature of 40° C. overnight. 250 ml of this solution are diluted 5-fold in 0.1 M acetic acid then reconcentrated to 500 ml by tangential filtration using the Sartoflow® Smart device with two Sartocon® Slice PESU cassettes (polyethersulfone membranes; cutoff threshold: 100 kDa; filtration surface area: 0.02 m²). The filtration is continued at a constant concentration against 5 l of acetic acid and 2.5 l of ultrapure water, then the solution is reconcentrated to 250 ml.

In the case of the MEX-CDFO (83), the iron chelation method explained above is also used with the aim of estimating the DS. This method makes it possible to estimate the DS of MEX-CDFO (B3) at 3%.

1H NMR analysis makes it possible to confirm the grafting of the DFO and the DS by virtue of the peaks characteristic of DFO. According to the NMR spectrum, the DS is equal to 2.9%. It is also possible to determine the DA with this same spectrum. The DA found is 41%.

### Example 4—Compositions According to the Invention (Aqueous Solutions)

Different compositions according to the invention were prepared. A non-functionalized chitosan A with a low DA, which may be 250 kDa or 650 kDa, is mixed with a copolysaccharide B in a ratio α/β% in which a is the concentration by weight of the chitosan A and β is the concentration by weight of the copolysaccharide B in the formulation in question (table 2). Different amounts of chitosan A (α) and of copolysaccharide B (β) in lyophilized form are introduced into a 50 ml reactor and are redispersed with gentle stirring in a suitable volume of water. Acetic acid is added in a stoichiometric proportion relative to the non-functionalized primary amine functions present in the medium. The mixture is left under stirring until the product has completely dissolved.

By way of example, composition 10 according to the invention (HG-5-10) is prepared as follows: 3.0 g of B1 (MEX-CD2) and 1.5 g of chitosan A1 are dispersed in 28.93 ml of ultrapure water and 1.07 ml of ultrapure acetic acid are added to a 50 ml reactor with mechanical stirring at 100 rpm. The mixture is left under stirring for 24 h, until it has completely dissolved and the medium is homogenized.

The different compositions and the preparations thereof are presented in table 2.

TABLE 2
Amounts of reagents applied during the synthesis of the different
compositions

| | | Concen- | | | |
|---|---|---|---|---|---|
| | | tration | Weight | | |
| | | by | of | Volume | Volume |
| | | weight | product | H₂O | CH₃COOH |
| Composition | Products | (g/l) | (g) | (ml) | (ml) |
| 1 (comparative) | Chitosan | 50 | 1.5 | 29.51 | 0.49 |
| (Ref HG-5-0) | 650 | | | | |
| | A2 | | | | |
| 2 (comparative) | Chitosan | 80 | 2.4 | 29.22 | 0.78 |
| (Ref HG-8-0) | 250 | | | | |
| | A1 | | | | |
| 3 (comparative) | Chitosan | 120 | 3.6 | 28.83 | 1.17 |
| (Ref HG-12-0) | 250 | | | | |
| | A1 | | | | |
| 4 (comparative) | Chitosan | 30 | 0.9 | 29.41 | 0.59 |
| (Ref HG-3-3) | 650 | | | | |
| | A2 | | | | |
| | Chitosan | 30 | 0.9 | | |
| | 250 | | | | |
| | A1 | | | | |
| 5 (comparative) | Chitosan | 40 | 1.2 | 29.22 | 0.78 |
| (Ref HG-4-4) | 650 | | | | |
| | A2 | | | | |
| | Chitosan | 40 | 1.2 | | |
| | 250 | | | | |
| | A1 | | | | |
| 6 (comparative) | MEX-CD2 | 100 | 3 | 29.42 | 0.58 |
| (HG-0-10) | B1 | | | | |
| 7 (comparative) | MEX-CD2 | 150 | 4.5 | 29.13 | 0.87 |
| (HG-0-15) | B1 | | | | |
| 8 (comparative) | MEX-CD2 | 200 | 6 | 28.84 | 1.16 |
| (HG-0-20) | B1 | | | | |
| 9 (inventive) | Chitosan | 50 | 1.5 | 29.22 | 0.78 |
| (HG-5-5) | 250 | | | | |
| | A1 | | | | |
| | MEX-CD2 | 50 | 1.5 | | |
| | B1 | | | | |
| 10 (inventive) | Chitosan | 50 | 1.5 | 28.93 | 1.07 |
| (HG-5-10) | 250 | | | | |
| | A1 | | | | |
| | MEX-CD2 | 100 | 3 | | |
| | B1 | | | | |
| 11 (inventive) | Chitosan | 50 | 1.5 | 28.64 | 1.36 |
| (HG-5-15) | 250 | | | | |
| | A1 | | | | |
| | MEX-CD2 | 150 | 4.5 | | |
| | B1 | | | | |
| 12 (inventive) | Chitosan | 80 | 2.4 | 28.75 | 1.25 |
| (HG-8-8) | 250 | | | | |
| | A1 | | | | |
| | MEX-CD2 | 80 | 2.4 | | |
| | B1 | | | | |
| 13 (inventive) | Chitosan | 30 | 0.9 | 29.53 | 0.47 |
| (HG-3-3) | 650 | | | | |
| | A2 | | | | |
| | MEX-CD2 | 30 | 0.9 | | |
| | B1 | | | | |
| 14 (inventive) | Chitosan | 40 | 1.2 | 29.38 | 0.62 |
| (HG-4-4) | 650 | | | | |
| | A2 | | | | |
| | MEX-CD2 | 40 | 1.2 | | |
| | B1 | | | | |
| 15 (inventive) | Chitosan | 50 | 1.5 | 28.94 | 1.06 |
| (HG-5-9-1) | 250 | | | | |
| | A1 | | | | |
| | MEX-CD2 | 90 | 2.7 | | |
| | B1 | | | | |
| | MEX- | 10 | 0.3 | | |
| | CDDFO1 | | | | |
| | B2 | | | | |
| 16 (inventive) | Chitosan | 56 | 1 | 17 | 0.72 |
| (HG-5-9.5-0.5) | 250 | | | | |
| | A1 | | | | |
| | MEX-CD2 | 107 | 1.9 | | |
| | B1 | | | | |
| | MEX-CDFO | 6 | 0.1 | | |
| | B3 | | | | |
| 17 (inventive) | Chitosan | 56 | 1 | 17 | 0.72 |
| (HG-5-9.2-0.8) | 250 | | | | |
| | A1 | | | | |
| | MEX-CD2 | 104 | 1.84 | | |
| | B1 | | | | |
| | MEX-CDFO | 9 | 0.16 | | |
| | B3 | | | | |
| 18 (inventive) | Chitosan | 56 | 1 | 17 | 0.72 |
| (HG-5-8-2) | 250 | | | | |
| | A1 | | | | |
| | MEX-CD2 | 90 | 1.6 | | |
| | B1 | | | | |
| | MEX-CDFO | 23 | 0.4 | | |
| | B3 | | | | |

The solution obtained is recovered and introduced into a suitable fluid metering device, then centrifuged at 4000 rpm for 1 minutes in order to obtain a solution with no air bubbles and comprising the composition. The Newtonian viscosities for each solution were determined using an AR200 rheometer from TA Instruments. Each solution obtained was also tested to ascertain whether it was possible to produced a gel and/or a thread. The results are presented below (table 3).

TABLE 3
Summary of the compositions and their physico-chemical characteristics

| | | Concen- | | | |
|---|---|---|---|---|---|
| | | tration | | | |
| | | by | Gelling | Thread | Newtonian |
| | | weight | in | formation | viscosity |
| Composition | Products | (g/l) | NaOH | possible | (Pa · s) |
| 1 (comparative) | Chitosan | 50 | Yes | Yes | 23058 |
| (Ref HG-5-0) | 650 | | | | |
| 2 (comparative) | Chitosan | 80 | Yes | Yes | 782 |
| (Ref HG-8-0) | 250 | | | | |
| 3 (comparative) | Chitosan | 120 | Yes | Yes | 27098 |
| (Ref HG-12-0) | 250 | | | | |
| 4 (comparative) | Chitosan | 30 | Yes | Yes | 9261 |
| | 650 | | | | |
| | 250 | | | | |
| 5 (comparative) | Chitosan | 40 | Yes | Yes | 19696 |
| (Ref HG-4-4) | 650 | | | | |
| | Chitosan | 40 | | | |
| | 250 | | | | |
| 6 (comparative) | MEX-CD2 | 100 | No | No | 52 |
| (HG-0-10) | | | | | |
| 7 (comparative) | MEX-CD2 | 150 | No | No | 1095 |
| (HG-0-15) | | | | | |
| 8 (comparative) | MEX-CD2 | 200 | No | No | 1861 |
| (HG-0-20) | | | | | |
| 9 (inventive) | Chitosan | 50 | Yes | No | 450 |
| (HG-5-5) | 250 | | | | |
| | MEX-CD2 | 50 | | | |
| 10 (inventive) | Chitosan | 50 | Yes | Yes | 2438 |
| (HG-5-10) | 250 | | | | |
| | MEX-CD2 | 100 | | | |
| 11 (inventive) | Chitosan | 50 | Yes | Yes | 8059 |
| (HG-5-15) | 250 | | | | |
| | MEX-CD2 | 150 | | | |
| 12 (inventive) | Chitosan | 80 | Yes | Yes | 7211 |
| (HG-8-8) | 250 | | | | |
| | MEX-CD2 | 80 | | | |
| 13 (inventive) | Chitosan | 30 | Yes | Yes | 3297 |
| (HG-3-3) | 650 | | | | |
| | MEX-CD2 | 30 | | | |
| 14 (inventive) | Chitosan | 40 | Yes | Yes | 19456 |
| (HG-4-4) | 650 | | | | |
| | MEX-CD2 | 40 | | | |
| 15 (inventive) | Chitosan | 50 | Yes | Yes | 2640 |
| (HG-5-9-1) | 250 | | | | |
| | MEX-CD2 | 90 | | | |
| | MEX- | 10 | | | |
| | CDDFO1 | | | | |

These results show that, unlike the composition according to the invention, when the composition comprises solely copolysaccharide B, it is not possible to gel the solution or to produce threads. However, the addition of a copolysaccharide B to a chitosan A makes it possible to obtain a gellable solution and, in some cases, to lower the viscosity of the resulting solution (cf. comparative compositions 2 and 3 and composition 9, and comparative composition 4 and composition 13), which contributes to improving the manageability of the solution and enables better thread formation.

### Example 5—Compositions According to the Invention (Gels)

A plurality of hydrogels were obtained by gelling solutions obtained in example 3. The gelling of the solution takes place after submerging in a 3 mol/l sodium hydroxide bath. The hydrogels obtained according to the invention are characterized as physical hydrogels in which gelling takes place at basic pH after neutralization of the amines (NH₂) which were initially protonated (NH₃⁺) present in the solution. The entangled state of the chains resulting from the high initial viscosity is fixed by the formation of inter-chain physical interaction nodes and thereby provides a stable physical hydrogel having adequate mechanical properties to be handled or stretched. The resulting hydrogels are reversible by treatment in acid solution, and differ from chemical hydrogels in which the gelling is provided by the formation of irreversible covalent chemical bonds. They can be reversibly dried and rehydrated.

### Obtaining Hydrogels in Disk Form

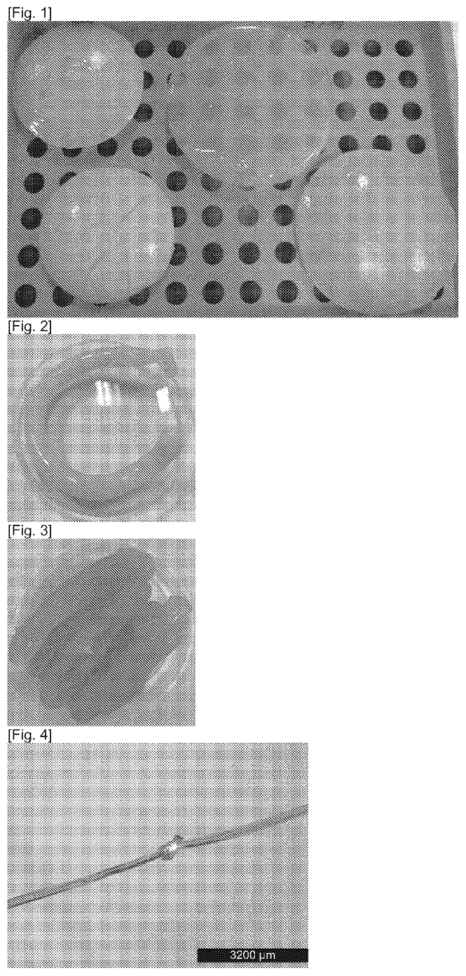

After centrifugation, compositions 9 to 12 according to the invention are introduced into a PVC mold, the dimensions of which are either Ø=1 cm and h=2 mm or Ø=3 cm and h=2.5 mm. After the solution has spread out uniformly, the mold containing the solution is introduced into a 3 mol/l sodium hydroxide (NaOH) bath for 1 h 30. The hydrogel disk thus formed is removed from the mold and introduced into a water bath for 30 minutes. The disk is subsequently rinsed a second time in another bath of ultrapure water for 30 minutes. The hydrogel is subsequently rinsed in a phosphate buffer at pH=7.4 for 24 hours. It is subsequently introduced with phosphate buffer into a glass vial and sterilized for 20 minutes at 121° C. in an autoclave. The hydrogels obtained in disk form are presented in FIG. 1.

### Obtaining Hydrogel in Cylindrical Form

After centrifugation, comparative composition 2 and composition 10 according to the invention are connected to a compressed air system (Nordson Ultimus™) enabling the solution to be extruded. An extrusion tube 5 mm in diameter is adapted to the syringe of the dispenser and the solution is extruded directly into a 3 mol/l sodium hydroxide bath. The extrusion pressure is adapted to the initial viscosity of the precursor solution and is between 1 and 3 bar. The hydrogel thus formed is left submerged in the sodium hydroxide bath for 1 h 30. The disk is removed and introduced into a bath of ultrapure water for 30 minutes. The tube is subsequently rinsed a second time in another bath of ultrapure water for 30 minutes. The hydrogel is subsequently rinsed in a phosphate buffer at pH 7.4 for 24 hours. It is finally introduced with phosphate buffer into a glass vial and sterilized for 20 minutes at 121° C. in an autoclave. The hydrogels obtained in cylindrical form are presented in FIGS. 2 and 3.

The hydrogel obtained from chitosan A alone has an opaque white appearance and is relatively rigid when handled. The more the hydrogel is loaded with copolysaccharide B, the more transparent the hydrogel appears, with a less pronounced Tyndall effect reflecting improved homogeneity of the structure on a microscopic level.

### Example 6—Compositions According to the Invention (Threads)

Threads comprising the composition according to the invention are obtained as follows: some of the solutions obtained in example 3 are introduced into a suitable fluid dispenser (or metering device), and are then centrifuged. The fluid dispenser containing the solution is subsequently connected to a compressed air system. The dispenser is equipped with an extrusion cone, which may be 254 μm, 406 μm, or 584 μm, then the solution is extruded under a constant pressure, which may range from 150 kPa to 400 kPa, into a concentrated 3 mol/l sodium hydroxide bath. The hydrogel obtained is neutralized in two water baths then dried at a temperature which may range from 100° C. to 200° C. using a temperature-regulated air blower. The dry thread is recovered and wound into reels.

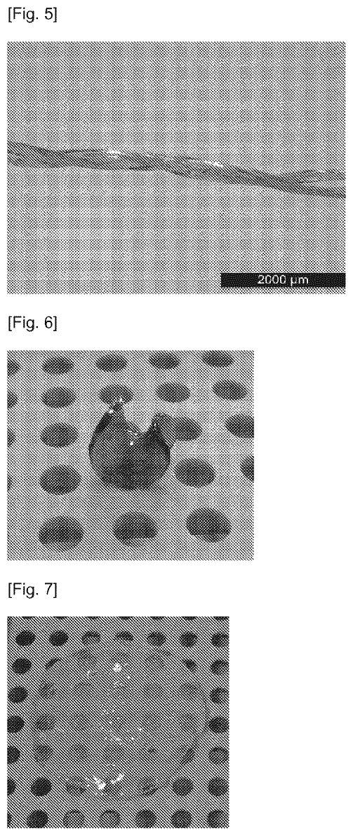

By way of example, a thread of composition 10 according to the invention is prepared as follows: 3.0 g of copolysaccharide B1 and 1.5 g of chitosan A1 are dispersed in 28.93 ml of ultrapure water and 1.07 ml of ultrapure acetic acid in a 50 ml reactor with gentle mechanical stirring at 100 rpm. The mixture is left under stirring for 24 h, until it has completely dissolved and the medium is homogenized. The solution obtained is recovered and introduced into a suitable fluid dispenser, then centrifuged at 4000 rpm for 10 minutes. The fluid dispenser containing the solution is subsequently connected to a compressed air system, and the system is equipped with a 406 μm extrusion cone. A constant pressure of 200 kPa is delivered until the starting solution has been completely extruded. The solution is extruded directly into a 3 mol/I sodium hydroxide bath and gelled directly to form a hydrogel. The hydrogel obtained in the form of a thin cylinder is passed into a reeling circuit and is neutralized gradually in two ultrapure water baths, each containing 5 l of water. The hydrogel is subsequently dried under hot air at 150° C. using a temperature-regulated air blower. The thread obtained after drying is wound onto a reel located at the end of the thread-forming circuit. The thread obtained is presented in FIGS. 4 and 5. These figures show that it is possible to make knots and to braid a plurality of threads together.

The mean diameter of the threads was determined using an optical microscope by measuring different portions of the thread. On average, 5 portions of the thread were studied in order to establish a mean diameter which is as representative as possible of the overall structure. The linear density of the different threads was also determined by weighing different lengths of thread on a precision balance. The mechanical properties of the threads obtained were determined by tensile tests using a Shimadzu Autograph AG-X plus system. On average, five tensile tests are performed on each thread in order to verify the reproducibility of each measurement. The results are presented in table 4.

TABLE 4
Mechanical properties of threads resulting from a plurality of
compositions with different extrusion diameters

| | | | | | Modulus |
|---|---|---|---|---|---|
| | Extrusion | Thread | Linear | | of |
| | diameter | diameter | density | Tenacity | elasticity |
| Composition | (μm) | (μm) | (mg/m) | (cN/dTex) | (MPa) |
| 2 | 254 | 80 | 6.5 | 1.49 | 123.6 |
| (comparative) | 406 | 147.0 | 23 | 0.91 | 55.2 |
| (Ref HG-8-0) | | | | | |
| | 254 | 92.8 | 9 | 1.29 | 104.2 |
| 3 | 406 | 142.4 | 24.8 | 0.90 | 65.9 |
| (comparative) | | | | | |
| (Ref HG-12-0) | | | | | |
| 4 | 254 | 63 | 3.7 | 1.23 | 79.3 |
| (comparative) | 406 | 98 | 8.2 | 1.43 | 80 |
| (Ref HG-3-3) | | | | | |
| 12 (inventive) | 254 | 104.6 | 11.1 | 1.28 | 80.7 |
| (HG-8-8) | 406 | 193.2 | 30.4 | 1.02 | 27.1 |
| | 584 | 296.7 | 53.2 | 0.64 | 16.1 |
| 10 (inventive) | 254 | 96.3 | 8 | 1.26 | 63.9 |
| (HG-5-10) | 406 | 173 | 27.9 | 0.80 | 38.4 |
| | 584 | 209.5 | 73.6 | 0.53 | 33.4 |
| 11 (inventive) | 406 | 193.3 | 33.6 | 0.81 | 30.2 |
| (HG-5-15) | | | | | |
| 13 (inventive) | 254 | 69 | 3.5 | 1.64 | 75.9 |
| (HG-3-3) | 406 | 109.3 | 5.4 | 1.68 | 52.8 |
| | | | | De- | |
| | Extrusion | Elastic | Elastic | formation | Breaking |
| | diameter | deformation | strain | at break | strain |
| Composition | (um) | (%) | (MPa) | (%) | (MPa) |
| 2 | 254 | 0.69 | 82.1 | 2.7 | 193.7 |
| (comparative) | 406 | 0.71 | 34.9 | 9.2 | 123.4 |
| (Ref HG-8-0) | | | | | |
| 3 | 254 | 0.8 | 76.2 | 3.1 | 170.2 |
| (comparative) | 406 | 1.04 | 62.8 | 7.1 | 140.6 |
| (Ref HG-12-0) | | | | | |
| 4 | 254 | 0.98 | 69.3 | 8 | 147.3 |
| (comparative) | 406 | 0.7 | 51.7 | 6.1 | 156.4 |
| (Ref HG-3-3) | | | | | |
| 12 (inventive) | 254 | 0.84 | 63.9 | 9.3 | 165.4 |
| (HG-8-8) | 406 | 1.85 | 43.2 | 13.7 | 106.1 |
| | 584 | 0.98 | 14.3 | 15.3 | 48.9 |
| 10 (inventive) | 254 | 0.97 | 54.7 | 7.6 | 139 |
| (HG-5-10) | 406 | 0.94 | 32.6 | 12.7 | 94.8 |
| | 584 | 1.08 | 31.9 | 19.1 | 114.2 |
| 11 (inventive) | 406 | 1.22 | 34.5 | 16.5 | 92.7 |
| (HG-5-15) | | | | | |
| 13 (inventive) | 254 | 0.77 | 57.5 | 8.1 | 153.9 |
| (HG-3-3) | 406 | 0.71 | 35.4 | 5.5 | 96.1 |

These results show that the threads according to the invention have satisfactory mechanical properties which are comparable to those of the threads of chitosan A alone. In particular, the threads according to the invention have suitable mechanical properties for shaping the threads by braiding a plurality of threads or by weaving or knitting.

### Example 7—Study of the Swelling of the Threads and Gels According to the Invention

The gels and threads according to the invention, described in the preceding sections, all have unique swelling abilities in the presence of aqueous fluids. Firstly, the capacity and the kinetics of drying and swelling of the gels were determined on the basis of the initial formulation. After neutralizing the pH of the hydrogels by successive rinsing, the gel is left in the open air for 1 week until it has completely dried into a xerogel. The weight of the hydrogel was measured using a precision balance before drying and at regular time intervals during the drying. The drying kinetics of the hydrogels is listed in table 6. Since the chitosan A is more hydrophobic than the copolysaccharides B, the drying rate is slower when the amount of copolysaccharide B contained in the gel is high. Thus, the reference gel of chitosan A alone (comparative composition 2) reaches a stable weight after 30 hours, composition 9 according to the invention (HG-5-5) after 52 hours, composition 12 according to the invention (HG-8-8) after 72 h, and compositions 10 and 11 according to the invention (HG-5-10 et HG-5-15) after 144 hours.

TABLE 5
Drying kinetics of the hydrogels

| | Percentage by weight of the hydrogel relative to its | | | | | |
|---|---|---|---|---|---|---|
| | initial weight, on the basis of the drying duration | | | | | |
| Composition | 3 h | 7 h | 24 h | 48 h | 144 h | 168 h |
| 2 (comparative) | 87.3% | 61.6% | 11.0% | 9.9% | 9.8% | 9.8% |
| (Ref HG-8-0) | | | | | | |
| 9 (inventive) | 89.8% | 79.4% | 38.8% | 5.9% | 4.7% | 4.7% |
| (HG-5-5) | | | | | | |
| 12 (inventive) | 90.7% | 81.8% | 47.3% | 12.9% | 7.6% | 7.6% |
| (HG-8-8) | | | | | | |
| 10 (inventive) | 92.1% | 84.5% | 54.1% | 17.7% | 3.9% | 3.9% |
| (HG-5-10) | | | | | | |
| 11 (inventive) | 91.7% | 83.7% | 52.3% | 16.5% | 4.0% | 4.0% |
| (HG-5-15) | | | | | | |

The xerogels were subsequently submerged in ultrapure water for 48 hours until the gels were completely hydrated. The weight of each gel was precisely measured before hydration and at regular time intervals during the swelling test. The swelling kinetics of the gels is listed in table 6. In the same way as for the drying step, the hydrophobic nature of the reference gel based on chitosan A alone leads to limited rehydration of the gel. Thus, a low maximum swelling of the gel obtained from comparative composition 2 (Ref HG-8-0), of approximately twice its initial volume, is observed after 6 hours. On the other hand, a strong increase in the swelling capacity of the gels according to the invention is observed on the basis of the amount of copolysaccharide B present in the formulation, ranging up to more than 22 times its initial volume for composition 11 according to the invention (HG-5-15) after 30 hours.

The gels before and after hydration are presented in FIGS. 6 and 7 (composition 11 according to the invention).

TABLE 6
Swelling kinetics of the gels during rehydration

| | Percentage by weight of the gel relative | | | | | |
|---|---|---|---|---|---|---|
| | to its initial weight, on the basis of the | | | | | |
| | duration of submersion in ultrapure water | | | | | |
| Composition | 2 h | 4 h | 6 h | 8 h | 25 h | 50 h |
| 2 (comparative) | 156% | 183% | 188% | 192% | 195% | 202% |
| (Ref HG-8-0) | | | | | | |
| 9 (inventive) | 300% | 402% | 492% | 546% | 626% | 659% |
| (HG-5-5) | | | | | | |
| 12 (inventive) | 252% | 403% | 555% | 671% | 941% | 977% |
| (HG-8-8) | | | | | | |
| 10 (inventive) | 373% | 713% | 984% | 1210% | 1803% | 1907% |
| (HG-5-10) | | | | | | |
| 11 (inventive) | 662% | 1301% | 1761% | 1978% | 2190% | 2237% |
| (HG-5-15) | | | | | | |

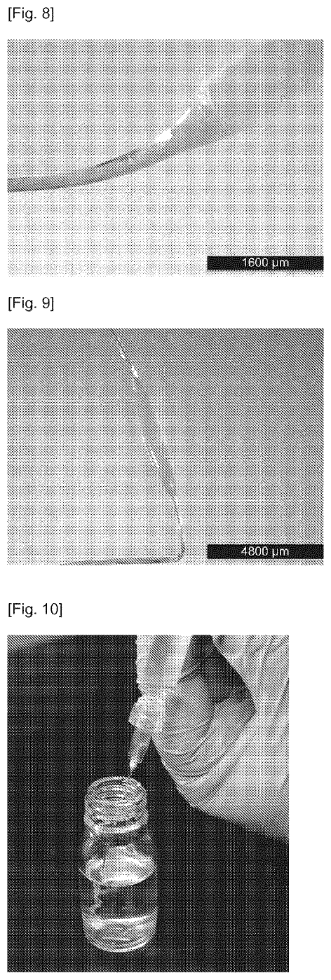

The swelling capacity of the threads according to the invention was also evaluated during their rehydration by being submerged in a bath of ultrapure water. In the same way as for the solid hydrogels, threads according to the invention were cut up then submerged in ultrapure water for 15 minutes. The initial weight of each thread, and the weight of the thread at regular time intervals, were measured using a precision balance. The swelling kinetics for the threads is listed in table 7. A slight difference in swelling is noted for a given formulation on the basis of the initial diameter of the thread. For an identical formulation, the swelling values obtained from the threads are relatively similar to those obtained with the hydrogels. In the same way as for the hydrogels, the reference thread based on chitosan A (comparative composition 2, ref HG-8-0) exhibits a low maximum swelling of approximately twice its initial volume after 15 minutes. The thread obtained with the composition 11 according to the invention (HG-5-15), containing the most copolysaccharide B, exhibits a swelling capacity equivalent to the hydrogel of the same formulation, ranging up to 21 times its initial volume after being submerged for 15 minutes. Unlike the hydrogels, the rehydration observed with the threads is very quick and results from a much higher surface-to-volume ratio for the threads than for the solid hydrogels. The phenomenon of swelling of the threads was also observed microscopically by submerging part of a thread obtained with composition 11 according to the invention (HG-5-15) in ultrapure water for 5 minutes. In the same way, the influence of a drop of water deposited on a portion of thread was observed. The threads are presented in FIGS. 8 and 9.

These results show that the compositions according to the invention have a much higher swelling capacity than the threads with only chitosan A.

TABLE 7
Swelling kinetics of the threads during rehydration

| | Thread | Percentage by weight of the thread relative | | | | |
|---|---|---|---|---|---|---|
| | diam- | to its initial weight, on the basis of the | | | | |
| | eter | duration of submersion in ultrapure water | | | | |
| Composition | (μm) | 1 min | 2 min | 5 min | 10 min | 15 min |
| 2 (comparative) | 80 | 147% | 178% | 200% | 229% | 220% |
| (Ref HG-8-0) | 147.0 | 153% | 160% | 171% | 212% | 209% |
| 12 (inventive) | 104.6 | 1227% | 1347% | 1320% | 1300% | 1355% |
| (HG-8-8) | 193.2 | 661% | 1207% | 1239% | 1218% | 1257% |
| | 296.7 | 582% | 808% | 832% | 927% | 958% |
| 10 (inventive) | 96.3 | 1230% | 1800% | 1823% | 1869% | 1882% |
| (HG-5-10) | 173 | 1012% | 1516% | 1978% | 1997% | 1983% |
| | 209.5 | 653% | 1125% | 1357% | 1648% | 1675% |
| 11 (inventive) | 193.3 | 574% | 1015% | 1797% | 2139% | 2134% |
| (HG-5-15) | | | | | | |

An experiment was performed on a disk of hydrogel according to the invention in order to evaluate the influence of a plurality of drying-swelling cycles on the hydration and dehydration capacities of the hydrogel. A solution of composition 11 according to the invention (HG-5-15) was formulated then gelled in a mold having the dimensions Ø=3 cm and h=2.5 mm in order to obtain a hydrogel in disk form, the weight of which was measured precisely using the precision balance. The hydrogel is subsequently rinsed as described above, then left to dry for 1 week and weighed. The xerogel is then hydrated again and weight after complete hydration, then dried again for a week. After one week, the gel is weighed then submerged in water again for 5 days. The values obtained over a plurality of cycles are listed in table 8.

TABLE 8
Swelling and drying capacity of the hydrogel disk obtained with
composition 11 according to the invention during hydration/
dehydration cycles

| | Weight of hydrogel in grams, on the basis of the | | | | |
|---|---|---|---|---|---|
| | hydration or dehydration time | | | | |
| | | t = 2 days | t = 7 days | t = 9 days | t = 14 days |
| Composition | t = 0 | (1^{st} drying) | (1^{st} swelling) | (2^{nd} drying) | (2^{nd} swelling) |
| 10 | 8.19 | 0.31 | 7.75 | 0.28 | 7.09 |
| (inventive) | | | | | |
| (HG-5-15) | | | | | |

A small loss of weight associated with a reduction in the swelling and drying capacities of the gel appears to gradually arise over the hydration and dehydration cycles, but it remains very limited. Likewise, a limited loss of 14% of the swelling capacities is observed after two full cycles of hydration and dehydration spread over a total of two weeks.

### Example 8—Study of the Metal Extraction Capacity of the Threads According to the Invention

The capacity of certain threads according to the invention to extract lead, copper, cadmium and iron was evaluated precisely by ICP-MS analysis after submerging the threads in diluted solutions of metals. Firstly, an experiment was performed on a metal solution containing three metal cations (Cu²⁺, Pb²⁺, Cd²⁺). 10 ml of metal solutions composed of the three metals Cu, Pb and Cd at 0 ppb, 10 ppb and 100 ppb, respectively, were prepared in ultrapure water from reference solutions of each metal at 500 ppb. In parallel, a 1 mg thread is weighed precisely on the precision balance, and this piece is then cut into four pieces of identical length and weight (weight=0.25 mg). The four thread pieces are subsequently submerged in each metal solution. After a duration of 4 hours, 1 day, 2 days and a week, respectively, a thread piece is collected and dried in an oven for 24 h at 40° C. The dry samples at 4 hours, 1 day and 2 days are subsequently analyzed using an ICP-MS Nexion 2000 spectrophotometer from Perkin-Elmer without prior mineralization and after simply dissolving the dry thread in 1 ml of a 10 wt % nitric acid (HNO₃) solution. The samples at 1 week are recovered then introduced into a Teflon container with 2 ml of 69 wt % HNO₃ and 1 ml of ultrapure water for digestion in a Multiwave 5000 microwave from Anton Paar for 30 Minutes at 200° C. The mineralized product is subsequently analyzed by ICP-M. The results obtained for the thread obtained with composition 12 according to the invention (HG-8-8) are presented in table 9.

TABLE 9
Metal extraction capacity of the thread obtained with composition 12
according to the invention (HG-8-8), kinetics and competition
between Pb²⁺, Cd²⁺ and Cu²⁺ ions

| | | | |
|---|---|---|---|
| Initial | Amount of | Amount of | |
| concentration | copper | cadmium | Amount of |
| of metal | extracted | extracted in | lead extracted |
| solution | in the dry | the dry thread | in the dry |
| (ppb) | thread (ppm) | (ppm) | thread (ppm) |

Extraction capacity after submerging the thread in the metal solution
for 4 hours with samples obtained without mineralization

| | | | |
|---|---|---|---|
| 0 | 1.3 | 0.61 | 0.39 |
| 10 | 4.32 | 2.19 | 6.42 |
| 100 | 57.67 | 67.74 | 5.95 |

Extraction capacity after submerging the thread in the metal solution
for 1 week with samples obtained after mineralization

| | | | |
|---|---|---|---|
| 0 | 5.84 | 0.53 | 0.25 |
| 10 | 15.95 | 1.39 | 2.14 |
| 100 | 27.30 | 31.78 | 4.24 |

The thread used for this experiment is obtained with composition 12 according to the invention (HG-8-8) and is thus composed of 50% by weight of chitosan A and 50% by weight of copolysaccharide B. The thread was submerged in a metal solution corresponding to 10 000 times its own volume and is capable of effectively extracting copper, lead and cadmium, concentrating the metals more than 1000-fold in its volume compared to the surrounding solution. This thread is also capable of effectively extracting the three metals for dilute metal solutions of approximately ten ppb.

A metal extraction study was also performed in a biological medium on reconstituted and highly hemolyzed pig's blood in order to evaluate the iron extraction capacity of the threads. 10 ml of a solution of hemolyzed pig's blood are prepared by redispersing 10 g/l of dried powdered blood in ultrapure water. In parallel, a 1 mg thread is weighed precisely on the precision balance, and this piece is then cut into four pieces of identical length and weight (weight=0.25 mg). The four thread pieces are subsequently submerged in the reconstituted pig's blood solution. After a duration of 1 hour, 2 hours, 4 hours and 24 hours, respectively, a thread piece is collected then rinsed in 60 ml of ultrapure water for 1 minute before being dried in an oven for 24 h at 40° C. The dry threads are subsequently recovered then introduced into a Teflon container with 2 ml of 69 wt % HNO₃ and 1 ml of ultrapure water for digestion in a Multiwave 5000 microwave from Anton Paar for 30 minutes at 200° C. The mineralized product is subsequently analyzed by ICP-MS. The results obtained on three threads are presented in table 10.

TABLE 10
Iron extraction capacity of the threads according to the invention
compared to a thread of chitosan A alone, in hemolyzed pig's blood
reconstituted in pure water

| | Submersion | Amount of iron extracted in |
|---|---|---|
| Composition | time (h) | the dry thread (ppm) |
| 2 (comparative) | 1 | 233.1 |
| (HG-8-0) | 2 | 238.1 |
| 10 (Inventive) | 1 | 275.1 |
| (HG-5-10) | 2 | 346.8 |
| 15 (inventive) | 1 | 374.3 |
| (HG-5-9-1) | 2 | 362.5 |

After being submerged for an hour, the three threads are capable of extracting iron from the medium. However, the addition of copolysaccharide B to the composition makes it possible to increase the iron chelation capacities of the thread after one hour, and significantly so after 2 hours. This increase is particularly significant when the composition according to the invention comprises 2 different chelators of Rc type.

The iron extraction capacity according to the invention when faced with different iron(III) chelators (citrate, NTA, deferiprone) was evaluated precisely by ICP-MS analysis after submerging dots of xerogel in iron solutions (2 ppm of iron) containing iron chelators in proportions in order to have one iron cation per complex (11 citrate/1 iron; 1.1 NTA/1 iron; 4 deferiprone/1 iron). The solutions are buffered to pH 7.4 with 0.1 M PBS for solutions containing citrate or deferiprone; for solutions containing NTA, the buffer used is 0.05 M Tris HCl. A dot of xerogel of known weight was submerged in a volume of 20 ml of metal solution for a duration of 24 h in the case of the experiment with deferiprone and 90 h for the experiments with NTA and citrate. The control dots were submerged in a buffer solution (PBS or Tris HCl) for a time corresponding to the experiment in question. Samples were collected from the solutions at t: 0 and t: final. These samples are then analyzed using an ICP-MS Nexion 2000 spectrophotometer from Perkin-Elmer. The gel dots are recovered and mineralized in a Multiwave 5000 microwave from Anton Paar according to the method presented above. The resulting solutions are thus analyzed by ICP-MS. The results obtained for the inventions 10, 16, 17, 15, 18, having the compositions HG-5-1; HG-5-9,5-0,5; HG-5-9,2-0,8; HG-5-9-1; HG-5-8-2, respectively, are presented in table 11.

TABLE 11
Iron extraction capacity obtained with compositions (10, 16, 17, 15, 18)
faced with iron chelators (deferiprone, citrate and NTA)

| | | | |
|---|---|---|---|
| | | Initial iron | Amount of iron |
| | | concentration of | extracted per |
| | Weight of | metal solution | milligram of xerogel |
| Invention | xerogel (mg) | (ppb) | (μg/mg) |

Iron extraction capacity of xerogel faced with deferiprone in 24 h

| | | | |
|---|---|---|---|
| 10 (HG-5-10) | 31.0 | 2078 | 0.01 |
| 16 | 26.0 | | 0.14 |
| (HG-5-9.5-0.5) | | | |
| 17 | 27.0 | | 0.26 |
| (HG-5-9.2-0.8) | | | |
| 18 (HG-5-8-2) | 28.2 | | 0.41 |

Iron extraction capacity of xerogel faced with citrate in 90 h

| | | | |
|---|---|---|---|
| 10 (HG-5-10) | 30.9 | 1680 | 0.50 |
| 16 | 32.8 | | 0.55 |
| (HG-5-9.5-0.5) | | | |
| 17 | 25.2 | | 0.70 |
| (HG-5-9.2-0.8) | | | |
| 18 (HG-5-8-2) | 33.5 | | 0.63 |
| 15 (HG-5-9-1) | 29.2 | | 0.70 |

Iron extraction capacity of xerogel faced with NTA in 90 h

| | | | |
|---|---|---|---|
| 10 (HG-5-10) | 24 | 1932 | 1.22 |
| 16 | 30.9 | | 0.99 |
| (HG-5-9.5-0.5) | | | |
| 17 | 24.1 | | 1.24 |
| (HG-5-9.2-0.8) | | | |
| 18 (HG-5-8-2) | 28.9 | | 1.03 |
| 15 (HG-5-9-1) | 27.2 | | 1.07 |

The different compositions are capable of extracting iron from a solution comprising iron chelates having relatively low complex formation constants, such as citrate or NTA. In the event that the dots are faced with a strong iron complexing agent, as is the case for deferiprone, only gels comprising DFO, corresponding to inventions 16, 17, 15, 18, are capable of extracting the iron. Invention 18 (HG-5-8-2), having the most polymer MEX-CDFO (B3) in its composition, has the best iron extraction capacity when faced with deferiprone.

Another metal extraction study was performed in biological medium on bovine plasma in order to evaluate the iron extraction capacity of the dots under conditions close to reality. For inventions 10, 16, 17, 15 and 18, a dot was weighed and introduced into 20 ml of bovine plasma for 24 hours. The plasma at t: 0 is analyzed by ICP-MS. The dots are mineralized and analyzed by ICP-MS according to the method explained above. The results obtained for the inventions 10, 16, 17, 15, 18, having the compositions HG-5-10; HG-5-9,5-0,5; HG-5-9,2-0,8; HG-5-9-1; HG-5-8-2, respectively, are presented in table 12.

TABLE 12
Iron extraction capacity obtained with compositions
(10, 16, 17, 15, 18) in a biological medium (bovine plasma)
Iron extraction capacity of hydrogels in plasma after 24 h

| | Number | Initial iron | |
|---|---|---|---|
| | of | concentration of | Mean % iron |
| Invention | replicates | plasma (ppb) | extracted by the gel |
| 10 | 5 | 1887 | 3.0 |
| (HG-5-10) | | | |
| 16 | 3 | | 3.2 |
| (HG-5-9.5-0.5) | | | |
| 17 | 5 | | 3.2 |
| (HG-5-9.2-0.8) | | | |

After 24 hours, the 3 gels are capable of extracting a portion of the iron from the plasma.

### Example 9—Studies of the Release Capacity of the Threads and Hydrogels According to the Invention

The capacity to load the thread with a substance of interest and the controlled release thereof in another solution or a another medium was demonstrated. Firstly, the possibility of loading the thread with a substance and releasing it therefrom was evaluated using a fluorescent molecule. 10 ml of a 25 mg/I fluorescein solution is prepared by dissolving fluorescein in ultrapure water. A 4 mg thread is then cut up and weighed using the precision balance before it is submerged in the fluorescein solution for 10 minutes. The threads are subsequently removed from the solution and dried in an oven at 40° C. for 2 hours then at ambient temperature for 3 days. The thread is subsequently submerged in 20 ml of ultrapure water and a sample of 2 ml of this solution is collected after 0, 1, 2, 5, 10, 15, 30, 45 minutes and after 15 hours and is analyzed using a Cary Eclipse fluorescence spectrometer from Agilent (table 11).

TABLE 13
Evaluation of the release of fluorescein previously loaded into the
thread, demonstrated by fluorescence

| | Fluorescence intensity of the solution, associated with the | | |
|---|---|---|---|
| | release of fluorescein over time into ultrapure water (AU). | | |
| | | 12 | 10 |
| Time | 2 (comparative) | (inventive) | (inventive) |
| (min) | (HG-8-0) | (HG-8-8) | (HG-5-10) |
| 0 | 0 | 0 | 0 |
| 1 | 0.4 | 1.1 | 11.02 |
| 2 | 0.56 | 2.2 | 19.34 |
| 5 | 0.61 | 2.017 | 22.42 |
| 10 | 0.77 | 2.33 | / |
| 15 | 0.65 | 2.18 | 25.15 |
| 30 | 0.93 | 2.64 | 26.07 |
| 45 | 0.79 | 2.73 | / |
| 900 | 0.75 | 2.6 | 30.9 |

The capacities of three thread formulations in loading and releasing a substance of interest were studied using fluorescein, by analyzing the fluorescence of the solution after release. The three threads release fluorescein 1 minute after the loaded dry thread is submerged in ultrapure water. Comparative formulation 2 (HG-8-0), composed solely of chitosan A, releases very little fluorescein. The addition of copolysaccharide B to the thread composition makes it possible to increase the release capacity of the threads; this is particularly true the more the composition is loaded with copolysaccharide B.

An identical experiment was performed, this time loading the threads with miconazole, which is an imidazole-based antifungal molecule commonly used in various medical devices (topical spray, creams, lotions, etc.) to combat fungal infections. 100 ml of a 0.1 g/l miconazole solution is prepared by dissolving miconazole in ultrapure water. A 0.9 mg (10 cm) thread obtained from composition 10 according to the invention (HG-5-10) is then cut up and weighed using the precision balance before it is submerged in the miconazole solution for 10 minutes. The threads are then removed from the solution and suspended from a glass rod in order to dry for 3 hours at ambient temperature. The thread is subsequently submerged directly in 3 ml of ultrapure water contained in a plastic spectrophotometer cuvette, and the medium is analyzed at regular time intervals by fluorescence analysis using a Cary Eclipse spectrophotometer from Agilent (table 12).

TABLE 14
Evaluation of the release of miconazole previously loaded into the thread
obtained from composition 10 (HG-5-10), demonstrated by fluorescence

| | Fluorescence intensity of the solution, |
|---|---|
| | associated with the release of miconazole |
| Time (min) | over time into ultrapure water (AU). |
| 1 | 3 |
| 5 | 65 |
| 10 | 70 |
| 25 | 95 |
| 40 | 250 |
| 60 | 500 |
| 120 | 720 |

The same experiment was performed, loading the threads with penicillin, an antibiotic. 100 ml of a 0.1 g/l penicillin solution is prepared by dissolving penicillin in ultrapure water. A 0.9 mg (10 cm) thread obtained from composition 10 according to the invention (HG-5-10) is then cut up and weighed using the precision balance before it is submerged in the penicillin solution for 5 minutes. The threads are then removed from the solution and suspended from a glass rod in order to dry for 3 hours at ambient temperature. The thread is subsequently submerged in 10 ml of ultrapure water and samples are collected after 1 h, 2 h and 22 h. The samples are subsequently analyzed by UV-visible absorbance by means of a Varian Cary 50 UV-vis spectrophotometer from Agilent (table 13).

TABLE 15
Evaluation of the release of penicillin previously loaded into the thread
obtained from composition 10 (HG-5-10), demonstrated by
UV-visible absorbance at 200 nm

| | Absorbance at 200 nm of the solution, |
|---|---|
| | associated with the release of penicillin |
| Time (min) | over time into ultrapure water |
| 0 | 0 |
| 1 | 0.58 |
| 2 | 0.64 |
| 22 | 0.76 |

These results show that the compositions according to the invention can be loaded with active pharmaceutical ingredients and can also release said ingredients.

### Example 10—Preliminary Study of the Composition of a Gelling Injectable Solution

A plurality of solutions were prepared in the same way as described in example 4, but this time the amount of acetic acid is added so as to obtain a homogeneous mixture in which the acid is in a deficit relative to the non-functionalized amine functions. In brief, different amounts of chitosan A1 and compound B1 are added to a reactor with milli-Q water and acetic acid. The mixture is left under stirring for at least one hour at 100 rpm. The solution is subsequently recovered and introduced into a fluid dispenser then centrifuged at 4000 rpm for 10 minutes in order to remove the remaining air bubbles. The pH and osmolarity of each formulation were measured using a Mettler Toledo SevenCompact S210 pH meter and a Camlab Löser Micro MOD200 Plus osmometer, respectively. The different compositions and the preparations thereof are presented in table 16.

TABLE 16
Summary of the formulations and their physico-chemical
characteristics

| | | Concen- | Concen- | | |
|---|---|---|---|---|---|
| | | tration | tration | | |
| | | by | of acetic | | |
| | | weight | acid | | Osmolarity |
| Composition | Products | (g/kg) | (w/w) | pH | (mOsm/l) |
| 19 | MEX-CD2 | 100 | 0.72 | 4.59 | 315 |
| (comparative) | B1 | | | | |
| (IS-0-10) | | | | | |
| 21 | MEX-CD2 | 70 | 0.72 | 4.45 | 217 |
| (comparative) | B1 | | | | |
| (IS-0-7) | | | | | |
| 22 | Chitosan | 14 | 0.72 | 4.77 | 174 |
| (comparative) | 250 | | | | |
| | A1 | | | | |
| (IS-1.4-5.6) | MEX-CD2 | 56 | | | |
| | B1 | | | | |
| 23 | Chitosan | 16.7 | 0.72 | 4.78 | 118 |
| (comparative) | 250 | | | | |
| (Ref IS- | A1 | | | | |
| 1.7-3.3 | MEX-CD2 | 33.3 | | | |
| | B1 | | | | |
| 24 | Chitosan | 10 | 0.72 | 4.43 | 110 |
| (comparative) | 250 | | | | |
| (IS-1-2) | A1 | | | | |
| | MEX-CD2 | 20 | | | |
| | B1 | | | | |
| 25 | Chitosan | 15 | 0.72 | 4.71 | 109 |
| (comparative) | 250 | | | | |
| (IS-1.5-1.5) | A1 | | | | |
| | MEX-CD2 | 15 | | | |
| | B1 | | | | |
| 26 | MEX-CD2 | 100 | 0.36 | 4.82 | NA |
| (comparative) | B1 | | | | |
| (IS-0-10) | | | | | |
| 27 (inventive) | MEX-CD2 | 100 | 0 | 5.37 | NA |
| (IS-0-10) | B1 | | | | |
| 28 (inventive) | MEX-CD2 | 50 | 0 | 5.39 | 41 |
| (IS-0-5) | B1 | | | | |
| 29 | MEX-CD2 | 75 | 0 | 5.4 | 180 |
| (comparative) | B1 | | | | |
| (IS-0-7.5) | | | | | |

The pH values obtained for aqueous solutions of MEX-CD2 without the addition of acid are relatively low. This is explained by the prior presence of acetic acid in the MEX-CD2 lyophilizate to ensure the solubility thereof during the purification step. The acetic acid contained in the MEX-CD2 lyophilizate is also osmotically active and contributes to increasing the osmolarity.

The viscosity of each solution was measured using a HAAKE RheoStress 600 rheometer equipped with a C35/2° Ti L plate-cone geometry. The values of the Newtonian viscosities are presented in table 15. It is noted that the viscosity is directly dependent on the total polymer concentration; the lower the concentration, the lower the viscosity. In the same way, for the same total polymer concentration, a larger fraction by weight of MEX-CD2 contributes to reducing the viscosity.

The injectability of each composition was precisely measured using a Shimadzu AG-X plus force testing machine. Each solution was introduced into a 1 ml BD Hylok™ pre-fillable glass syringe equipped with a Sterican 27 G needle (0.4×12 mm). The injectability was determined as the force in Newtons required to eject the solution at a constant plunger rate of 1 mm/s. The injectability of the system mainly depends on the viscosity of the solution, and thus depends on the total polymer concentration and to a lesser extent on the fraction by weight of MEX-CD2 in the composition in question. Thus, the solutions are readily injectable at 3% (w/w), moderately injectable at 5% (w/w), difficult to inject at 7% (w/w) and cannot be injected at 10% (w/w) (T. E. Robinson et al. Filling the Gap: A Correlation between Objective and Subjective Measures of Injectability, Adv. Healthc. Mater., vol. 9, no 5, p. 1901521, 2020).

TABLE 17
Viscosity and injectability force correlated to pH and to the
composition of each sample

| | | Concen- | | | |
|---|---|---|---|---|---|
| | | tration | | | |
| | | by | | Newtonian | Ejection |
| | | weight | | viscosity | force |
| Composition | Products | (g/kg) | pH | (Pa · s) | (N) |
| 19 | MEX-CD2 | 100 | 4.59 | 36.8 | 96.5 |
| (comparative) | B1 | | | | |
| (Ref IS-0-10) | | | | | |
| 21 | MEX-CD2 | 70 | 4.45 | 4.33 | 41.3 |
| (comparative) | B1 | | | | |
| (IS-0-7) | | | | | |
| 22 | Chitosan | 14 | 4.77 | 17.5 | 59.1 |
| (comparative) | 250 | | | | |
| (IS-1.4-5.6) | A1 | | | | |
| | MEX-CD2 | 56 | | | |
| | B1 | | | | |
| | Chitosan | 16.7 | 4.78 | 5.12 | 32.6 |
| 23 (inventive) | 250 | | | | |
| (IS-1.7-3.3) | A1 | | | | |
| | MEX-CD2 | 33.3 | | | |
| | B1 | | | | |
| 24 | Chitosan | 10 | 4.43 | 0.565 | 14.1 |
| (comparative) | 250 | | | | |
| (IS-1-2) | A1 | | | | |
| | MEX-CD2 | 20 | | | |
| | B1 | | | | |
| 25 | Chitosan | 15 | 4.71 | 0.989 | 16.5 |
| (comparative) | 250 | | | | |
| (IS-1.5-1.5) | A1 | | | | |
| | MEX-CD2 | 15 | | | |
| | B1 | | | | |
| 26 | MEX-CD2 | 100 | 4.82 | 42.6 | 111.7 |
| (comparative) | B1 | | | | |
| (IS-0-10) | | | | | |
| 27 (inventive) | MEX-CD2 | 100 | 5.37 | 74.3 | 126.4 |
| (IS-0-10) | B1 | | | | |
| 28 (inventive) | MEX-CD2 | 50 | 5.39 | 1.8 | 31.8 |
| (IS-0-5) | B1 | | | | |
| 29 | MEX-CD2 | 75 | 5.4 | 19.5 | 62.6 |
| (comparative) | B1 | | | | |
| (IS-0-7.5) | | | | | |

### Example 11—Determining the Suitable Fraction by Weight of MEX-CD2 for a Gelling Injectable Solution

This example makes it possible to demonstrate the impact of the fraction by weight of MEX-CD2 on the final formulation. Consequently, 5% (w/v) solutions were produce with different fractions by weight of Mt-EX-CD2, ranging from 0% to 100% MEX-CD2. Each of these formulations was sterilized for 20 minutes at 121° C. in an autoclave in order to evaluate the impact of this method on the rheological properties of the system. The pH, osmolarity, viscosity and injectability of each of these formulations were determined and are presented in table 18.

TABLE 18
Osmolarity, viscosity and injectability force correlated to pH and
to the composition of each sample

| | | Concentration | | |
|---|---|---|---|---|
| | | by weight | | Osmolarity |
| Composition | Products | (g/l) | pH | (mOsm/l) |
| 30 | Chitosan | 50 | 5.29 | 211 |
| (comparative) | 250 | | | |
| (Ref IS-5-0) | A1 | | | |
| 31 | Chitosan | 44 | 5.05 | 199 |
| (comparative) | 250 | | | |
| (IS-4.4-0.6) | A1 | | | |
| | MEX-CD2 | 6 | | |
| | B1 | | | |
| 32 | Chitosan | 41 | 5.19 | 193 |
| (comparative) | 250 | | | |
| (IS-4.1-0.9) | A1 | | | |
| | MEX-CD2 | 9 | | |
| | B1 | | | |
| 33 | Chitosan | 37.5 | 5.21 | 182 |
| (comparative) | 250 | | | |
| (IS-3.8-1.2) | A1 | | | |
| | MEX-CD2 | 12.5 | | |
| | B1 | | | |
| 34 | Chitosan | 33.3 | 5.01 | 169 |
| (comparative) | 250 | | | |
| (IS-3.3-1.7) | A1 | | | |
| | MEX-CD2 | 16.7 | | |
| | B1 | | | |
| 35 | Chitosan | 25 | 4.77 | 199 |
| (comparative) | 250 | | | |
| (IS-2.5-2.5) | A1 | | | |
| | MEX-CD2 | 25 | | |
| | B1 | | | |
| 23 (inventive) | Chitosan | 16.7 | 4.56 | 212 |
| (IS-1.7-3.3) | 250 | | | |
| | A1 | | | |
| | MEX-CD2 | 33.3 | | |
| | B1 | | | |
| 36 | Chitosan | 12.5 | 4.46 | 224 |
| (comparative) | 250 | | | |
| (IS-2.5-2.5) | A1 | | | |
| | MEX-CD2 | 37.5 | | |
| | B1 | | | |
| 37 | Chitosan | 9 | 4.31 | 204 |
| (comparative) | 250 | | | |
| | A1 | | | |
| (IS-0.9-4.1) | MEX-CD2 | 41 | | |
| | B1 | | | |
| 38 | Chitosan | 6 | 4.33 | 222 |
| (comparative) | 250 | | | |
| | A1 | | | |
| (IS-0.6-4.4) | MEX-CD2 | 44 | | |
| | B1 | | | |
| 28 (inventive) | MEX-CD2 | 50 | 4.25 | 212 |
| (IS-0-5) | B1 | | | |
| | Fraction | Newtonian viscosity | | Ejection |
| | by weight | (Pa · s) | | force |
| | of | Before | After | (N) after |
| Composition | MEX-CD2 | sterilization | sterilization | sterilization |
| 30 | 0 | 14.68 | 5.76 | 37.67 |
| (comparative) | | | | |
| (Ref IS-5-0) | | | | |
| 31 | 0.13 | 13.28 | 8.78 | 38.07 |
| (comparative) | | | | |
| (IS-4.4-0.6) | | | | |
| 32 | 0.17 | 13.81 | 8.92 | 37.91 |
| (comparative) | | | | |
| (IS-4.1-0.9) | | | | |
| 33 | 0.25 | 13.01 | 8.19 | 37.42 |
| (comparative) | | | | |
| (IS-3.8-1.2) | | | | |
| 34 | 0.33 | 9.37 | 5.77 | 32.84 |
| (comparative) | | | | |
| (IS-3.3-1.7) | | | | |
| 35 | 0.5 | 5.44 | 4.02 | 30.45 |
| (comparative) | | | | |
| (IS-2.5-2.5) | | | | |
| 23 (inventive) | 0.67 | 2.88 | 2.55 | 29.44 |
| (IS-1.7-3.3) | | | | |
| 36 | 0.75 | 2.09 | 2.10 | 27.62 |
| (comparative) | | | | |
| (IS-2.5-2.5) | | | | |
| 37 | 0.83 | 0.733 | 0.531 | 19.28 |
| (comparative) | | | | |
| (IS-0.9-4.1) | | | | |
| 38 | 0.88 | 1.02 | 0.766 | 23.41 |
| (comparative) | | | | |
| (IS-0.6-4.4) | | | | |
| 28 (inventive) | 1 | 0.732 | 0.335 | 19.06 |
| (IS-0-5) | | | | |

These formulations were produced by adding acetic acid in stoichiometric proportions with the free amines of the two polymers; the residual amount of acetic acid present in the MEX-CD2 lyophilizate was not taken into consideration. This explains why the pH decreases virtually linearly with the amount of MEX-CD2 added to the composition and thus with the fraction by weight of MEX-CD2 of the formulation in question. The Newtonian viscosity, measured on these solutions before and after sterilization, decreases linearly as a function of the fraction by weight of MEX-CD2. The injectability follows the same trend and thus demonstrates that the greater the amount of MEX-CD2 in the formulation in question, the easier said formulation is to inject. The samples formulated at 5% (w/v) are all moderately injectable at 27 G with ejection forces ranging from 19 N for a formulation containing 100% MEX-CD2 to 38 N for a formulation containing 100% standard chitosan. The impact of the sterilization on the viscosity of the formulation is heavily dependent on the fraction by weight of MEX-CD2. Thus, the formulations comprising a fraction by weight of MEX-CD2 greater than or equal to 67% have viscosities which are not, or are only very slightly, adversely affected during the sterilization of the mixture.

### Example 12—Injectability and Gelling of a Gelling Injectable Solution MEX-CD2-I

This example aims to precisely determine the physico-chemical and rheological properties of two formulations of MEX-CD2-I (gelling injectable solution) selected from the results of the previous examples. Thus, the formulations at 5% (w/w) with a fraction by weight of MEX-CD2 equal to 67% and 100%, respectively, were studied more closely. The formulation containing 67% MEX-CD2 was synthesized at two slightly different pHs to evaluate the impact of the pH on the rheological properties of the formulation. Each of these two formulations was also prepared at 10% (w/w) in order to envisage mixing these more concentrated precursor solutions with molecules of interest at a 1:1 ratio. Each of these formulations was sterilized for 20 minutes at 121° C. in an autoclave in order to study the impact of sterilization on their properties. The pH, osmolarity and viscosity of each of these formulations were measured and are presented in table 19. For the 5% (w/w) formulations, injectability was also determined using different needles

TABLE 19
Osmolarity, viscosity before and after sterilization, and injectability force,
correlated to pH and to the composition of each sample

| | | Concentration | Concentration | | |
|---|---|---|---|---|---|
| | | by weight | of acetic acid | | Osmolarity |
| Composition | Products | (g/kg) | (w/w) | pH | (mOsm/l) |
| 23 | Chitosan | 16.7 | 0.36 | 5.85 | 85 |
| (inventive) | 250 | | | | |
| (IS-1.7-3.3) | A1 | | | | |
| | MEX-CD2 | 33.3 | 0.4 | 5.57 | 108 |
| | B1 | | | | |
| 28 | MEX-CD2 | 50 | 0 | 5.27 | 64 |
| (inventive) | B1 | | | | |
| (IS-0-5) | | | | | |
| 39 | Chitosan | 33.3 | 0.75 | 5.66 | 422 |
| (inventive) | 250 | | | | |
| (IS-3.3-6.7) | A1 | | | | |
| | MEX-CD2 | 66.7 | 2.26 | 4.53 | 610 |
| | B1 | | | | |
| 27 | MEX-CD2 | 100 | 0 | 5.51 | 184 |
| (inventive) | B1 | | | | |
| (IS-0-10) | | | | | |

| | | Newtonian viscosity | | Ejection force (N) | | |
|---|---|---|---|---|---|---|
| | | (Pa · s) | | Needles used (Ø × L in mm) | | |
| | | Before | After | 0.3 × 13 | 0.4 × 12 | 0.5 × 16 |
| Composition | pH | sterilization | sterilization | (30 G) | (27 G) | (25 G) |
| | 5.85 | 9.2 | 7.7 | 88.2 | 35.8 | 21.4 |
| 23 | 5.57 | 16.4 | 17.8 | NA | | |
| (inventive) | | | | | | |
| (IS-1.7-3.3) | | | | | | |
| 28 | 5.27 | 1.35 | 1.61 | 77.8 | 32.5 | 17.2 |

| | | | | |
|---|---|---|---|---|
| (inventive) | | | | |
| (IS-0-5) | | | | |
| 39 | 5.77 | 2023 | 3260 | NA |
| (inventive) | 4.53 | 236 | 359 | NA |
| (IS-3.3-6.7) | | | | |
| 27 | 5.51 | 148 | 296 | NA |
| (inventive) | | | | |
| (IS-0-10) | | | | |

The injectability of the formulations depends mainly on the Newtonian viscosity of the solution, on the internal radius of the needle and, to a lesser extent, on the length of the needle used. For a formulation at 5% (w/w) with a fraction by weight of MEX-CD2 of 67%, it is observed that a reduction in the pH generates a significant increase in injectability after sterilization of the mixture. This aligns with the observation made regarding the viscosity of the system. The formulation at 5% (w/w) with 100% MEX-CD2 is easier to inject than the formulation containing 67% MEX-CD2/CTS, regardless of which needle is used. Finally, regardless of the formulation envisaged, the system does not seem to be manually injectable with a 30 G needle. The two formulations can be injected manually by a practitioner with a 27 G or less needle.

Compositions IS-1,7-3,3 and IS-0-5 are able to gel spontaneously by being submerged in a physiological medium having a pH of greater than 6.2 and an osmolarity of greater than 285 mOsm/l [FIG. 10]. In order to evaluate the rheological properties of the gel formed, 2 ml of composition IS-7-3,3 are introduced into a disk-shaped mold 25 mm in diameter. The mold is submerged in 20 ml 10 mM PBS and left for 2 hours. The storage modulus (G′) and the loss modulus (G″) of the hydrogel formed were recorded on an ARES rheometer from TA Instruments, using a plate/plate geometry of 25 mm. Before the measurement, the apparatus is tared with the empty mold. A sweep at a constant deformation of 1% and a frequency of 10 Hz was performed on the hydrogel formed after the mold containing the hydrogel was placed on the geometry. The hydrogel begins to form spontaneously after submersion in PBS, and a progressive gelling front can be observed. The gel obtained has G′ and G″ values greater than 10² Pa for a high oscillation frequency, which corresponds to a very flexible gel. Moreover, the value of G′ is greater than that of G″, meaning that the resulting material is indeed closer to a gel than a solution. G′ and G″ decrease with frequency, which seems to demonstrate a significant relaxation phenomenon (flow/relaxation/mobility of the chains, viscoelastic effect) due to a weakly crosslinked gel.

### Example 13—Study of In Vivo Tolerance of Implantation of a Xerogel HG-5-10 in the Peritoneal Cavity

A xerogel (h=1.5 mm and 0=10 mm) in dot form was obtained by partially drying a gel comprising composition HG-5-10, prepared as described in example 4. This xerogel was implanted in the peritoneum of 10 male C57BI/6 mice in order to evaluate the biocompatibility and tolerance of the implant. The parameters evaluated in this study comprise observations and measurements during life (including morbidity/mortality, clinical signs, body weight, food consumption), hematology before and after treatment, and blood chemistry and the weight of the organs after treatment and autopsy. Half the mice were sacrificed on day 7 and the other half on day 56.

Under the experimental conditions employed, the xerogel HG-5-10 administered as a medical device which can be implanted in the peritoneum did not induce any signs of toxicity in male 057BI/6 mice. During this study, no deaths, significant changes to body weight or changes to the blood were induced in the mice by the implant, on day 7 and on day 56. The intra-abdominal implantation of the xerogel HG-5-10 did not cause any microscopic changes in the liver, nor any alterations to the weight of the liver.

### Example 14—In Vivo MRI Study of the Degradability of the Gelling Injectable Solution IS-1,7-3,3 by Subcutaneous Administration

Approximately 300 μl of formulation IS-1,7-3,3 loaded with 305 ppm of Gd (pre-complexed to MEX-CD2) were injected subcutaneously into the backs of healthy BALB/c mice using a 22 G syringe. The MRI images in FIG. 11 of the gel implanted in the mouse were acquired for 1 month following implantation, at 7.1 T on a 300 MHz Bruker Avance Neo spectrophotometer equipped with a micro 2.5 micro-imaging probe at ambient temperature (RT=21° C.). The signal intensities in the liver, kidneys and spleen were also monitored.

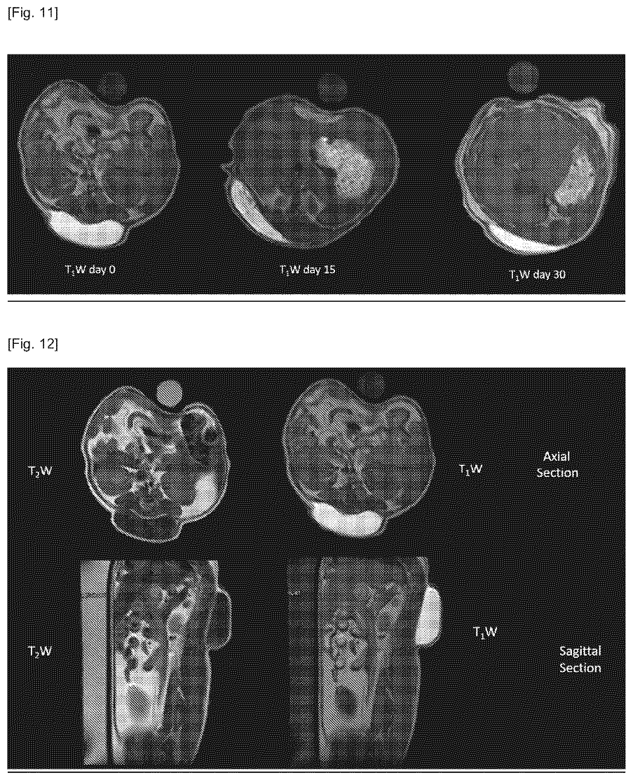

In FIG. 12, the T_{2w} images were acquired using a standard RARE (Rapid Acquisition with Refocused Echoes) sequence with the following parameters: TR=4000 ms, TE=24 ms, RARE factor=16, number of averages=4, FOV=30 mm, section thickness=1 mm, matrix size 128×128. The T_{1w} images were acquired using a standard MSME (multi spins multi echoes) sequence with the following parameters: TR=400 Ms, TE=3.3 ins, number of averages=6, FOV=30 mm, section thickness=1 mm, matrix size 128×128. The T₁ values were measured using a Saturation Recovery Spin Echo sequence (TE=3.8 ms, 10 TR variable, ranging from 50 to 5000 ms, FOV=30 mm, slice thickness=1 mm, matrix size 128×128) and analyzed using the T₁ saturation recovery equation. The size of the gel formed was measured by manually drawing the ROI on the MRI images.

As listed in table 20, the volume occupied by the gel formed decreased down to approximately 50% of the initial volume over 30 days of monitoring, while the T₁ measured in the same region increased approximately 6-fold. From day 15 following implantation, the heterogeneity of the implant began to increase due to cell infiltration. This infiltration is demonstrated by the increase in the T₁ standard deviations measured from day 15 to day 30. The starting volumes of the implants in the 3 mice were respectively 304, 348 and 216 mm³. The T_{1w} signal intensities in the liver, kidneys and spleen did not increase over the period of time studied.

TABLE 20
Variation in the volume of the hydrogel formed after injection of
formulation IS-1.7-3.3 after 1 month of monitoring, and
corresponding T₁ measured in the same region

| | Variation in volume (Vᵢ/V₀) | | T₁ (ms) | |
|---|---|---|---|---|
| Duration | | Standard | | Standard |
| (days) | Mean | deviation | Mean | deviation |
| 0 | 1 | NA | 58.0 | 2.71 |
| 1 | 0.804 | 0.07 | 61.5 | 2.38 |
| 2 | 0.757 | 0.05 | 86.3 | 3.59 |
| 4 | 0.71 | 0.05 | 131.3 | 3.3 |
| 8 | 0.656 | 0.06 | 238.5 | 12.07 |
| 11 | 0.633 | 0.06 | 333.5 | 15.3 |
| 15 | 0.582 | 0.04 | 333.8 | 45.5 |
| 18 | 0.561 | 0.03 | 330.5 | 42.1 |
| 22 | 0.533 | 0.04 | 286.3 | 36.8 |
| 26 | 0.516 | 0.04 | 282.2 | 41 |
| 30 | 0.499 | 0.04 | 276 | 41.9 |

1. A composition comprising:
a. at least one chitosan A, and
b. at least one statistical copolysaccharide B having a weight-average molecular weight of between 100 kDa and 1000 kDa, of formula I: 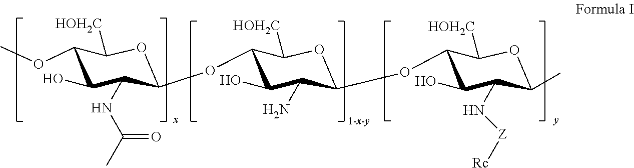
in which:
each Rc independently represents a group comprising a chelating agent,
each Z independently represents a linker which may be a single bond or a hydrocarbon-based chain comprising between 1 and 12 carbon atoms, it being possible for said chain to be linear or branched and to comprise one or more unsaturations and to comprise one or more heteroatoms,
x is between 0.01 and 0.5,
y is between 0.05 and 0.5,
the y/x ratio being greater than 0.2, preferably greater than 1,
the sum x+y being greater than 0.1, and
less than 10% of groups comprising a chelating agent of Rc type which are chelated by a cation of a transition element selected from block d and f elements, and
c. optionally water.

2. The composition as claimed in claim 1, characterized in that it has:
a degree of crystallinity, in relation to the chitosan A, of at least 10% relative to the total dry weight of the composition, and
a crystallite size of the chitosan A of less than 20 nm.

3. The composition as claimed in claim 1, characterized in that the weight ratio between A and B (A:B) is between 2:1 and 1:10; or between 1:1 and 1:5.

4. The composition as claimed in claim 1, characterized in that the chitosan A has an average molecular weight Mw of between 100 kg/mol and 1000 kg/mol or between 200 kg/mol and 700 kg/mol.

5. The composition as claimed in claim 1, characterized in that the chitosan A has a degree of acetylation x of less than 40% or less than 10% or between 0% and 10%.

6. The composition as claimed in claim 1, characterized in that the statistical copolysaccharide B having a weight-average molecular weight of between 100 kDa and 1000 kDa of formula I is of formula II: 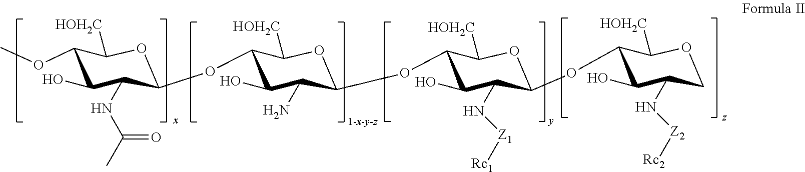
in which:
Rc1 and Rc2 are different and are groups comprising a chelating agent,
Z1 and Z2, which are identical or different, are linkers which may be a single bond or a hydrocarbon-based chain comprising between 1 and 12 carbon atoms, it being possible for said chain to be linear or branched and to comprise one or more unsaturations and to comprise one or more heteroatoms,
x is between 0.01 and 0.5, or between 0.01 and 0.1, or between 0.05 and 0.1,
y is between 0.01 and 0.5 or between 0.05 and 0.2,
z is between 0 and 0.2,
and
less than 10% of groups comprising a chelating agent of Rc1 and Rc2 type which are chelated by a cation of a transition element are selected from block d and f elements.

7. The composition as claimed in claim 1, characterized in that it additionally comprises at least one active pharmaceutical ingredient selected from anticancer agents, antibacterials, antifungals, anti-inflammatories, messenger RNAs, proteins and antigens.

8. The composition as claimed in claim 1, characterized in that it is an aqueous solution comprising at least 10 g/l of the mixture of chitosan A and of polysaccharide B.

9. The composition as claimed in claim 1, characterized in that it is in the form of a lyophilizate or a gel.

10. The composition as claimed in claim 9, characterized in that it is in the form of a xerogel, said xerogel resulting from the at least partial drying of a hydrogel.

11. The composition in xerogel form as claimed in claim 10, characterized in that, on contact with an aqueous medium or biological tissue for a period of at least 1 hour, it swells to form a hydrogel, said hydrogel having a volume at least twice as large, or at least 5 times as large, as the volume of the xerogel before contact with the aqueous medium or biological tissue.

12. A thread, characterized in that it comprises a gel or a lyophilizate of the composition as claimed in claim 9.

13. A textile material, characterized in that it comprises threads according to claim 12.

14. A medical device, characterized in that it comprises the composition as claimed in claim 1, the medical device being a dressing, an implant, or a dermal filler product.

15. The medical device as claimed in claim 14, characterized in that it is a dermal filler product in the form of a thread, said thread being in the form of a xerogel which can swell on contact with an aqueous medium or biological tissue in order to form a hydrogel.

16. A method of treating or preventing endometriosis, fungal infections, bacterial infections, chronic wounds, neurodegenerative disorders, nervous system lesions, hemochromatosis, Wilson's disease or lead poisoning in a subject in need thereof comprising implanting the composition of claim 1 into the subject, thereby capturing at least one metal in the subject.

17. A method for preparing a composition as claimed in claim 1, comprising the following steps:
1) dispersing the chitosan A and the statistical polysaccharide B having a weight-average molecular weight of between 100 kDa and 1000 kDa of formula I in water,
2) adding an acid,
3) mixing then centrifuging the solution obtained in order to recover a solution comprising the composition as claimed in claim 1,
4) optionally treating the solution obtained in step 3 in a basic aqueous bath in order to obtain the composition in gel form, and
5) optionally washing, drying and/or sterilizing the gel obtained in step 4.

18. The composition of claim 1, wherein the one or more heteroatoms is nitrogen, oxygen, sulfur or a halogen.

19. The composition of claim 6, wherein the one or more heteroatoms is nitrogen, oxygen, sulfur or a halogen.

20. The composition of claim 9, wherein the gel is a hydrogel, a xerogel or an aerogel.
